(19) United States
(12) Patent Application Publication
(10) Pub. No.: US 2024/0018220 A1
(43) Pub. Date: Jan. 18, 2024

(54) METHODS OF TREATING PATIENTS HAVING COMPLEMENT DISORDERS USING ANTI-C5 ANTIBODIES

(71) Applicant: Alexion Pharmaceuticals, Inc., Boston, MA (US)

(72) Inventors: Krista K. Johnson, Southington, CT (US); Paul P. Tamburini, Kensington, CT (US); Douglas L. Sheridan, Branford, CT (US)

(73) Assignee: Alexion Pharmaceuticals, Inc., Boston, MA (US)

(21) Appl. No.: 18/032,959

(22) PCT Filed: Oct. 22, 2021
(86) PCT No.: PCT/US2021/056153

§ 371 Date: Apr. 20, 2023

Related U.S. Application Data
(60) Provisional application No. 63/105,018, filed on Oct. 23, 2020.

(51) Int. Cl. C07K 16/18; G01N 33/536; G01N 30/02; G01N 21/47

(52) U.S. Cl. CPC C07K 16/18; G01N 33/536; G01N 30/02; G01N 21/47; G01N 2800/52; A61K 2039/507

(57) ABSTRACT

Provided herein are methods of treatment designed to prevent or minimize formation of deleterious multivalent immune complexes in a human patient having a complement mediated disorder (e.g., paroxysmal nocturnal hemoglobinuria (PNH) or atypical hemolytic uremic syndrome (aHUS)), who has been or is being treated with a first anti-C5 antibody and is then treated with a second (different) anti-C5 antibody, as well as methods of safely switching a patient from treatment with a first anti-C5 antibody to a second (different) anti-C5 antibody. Also provided are methods for determining an adjusted regimen antibody (e.g., a regimen to prevent or minimize formation of multivalent immune complexes) comprising an adjusted therapeutic dose and/or timing of administration of a second anti-C5 to treat a patient who has been or is being treated with a first anti-C5 antibody.

### CROSS REFERENCE TO RELATED APPLICATIONS

This application claims the benefit of U.S. Provisional Application No. 63/105,018, filed Oct. 23, 2020, the contents of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Oct. 8, 2021, is named 0641WO_SL.txt and is 58,786 bytes in size.

### BACKGROUND

The properties of circulating immune complexes formed with target antigens should be considered when developing a monoclonal antibody, e.g., for use in therapeutic applications. Valency of such complexes can affect clearance, effector function, and uptake by phagocytic cells. In the medical setting, the safety profiles of traditional monoclonal antibodies used in diagnosis and/or therapy of diseases may be change or evolve if they form multivalent immune complexes in vivo in the presence of secondary antibodies. Therefore, consideration must be given when a recipient is exposed to more than one antibody that binds to a monovalent target at different sites, potentially leading to the formation of multivalent immune complexes. Accordingly, it is an object of the present disclosure to provide improved methods for treating patients (e.g., patients undergoing treatment for complement mediated disorders, such as paroxysmal nocturnal hemoglobinuria (PNH) or atypical hemolytic uremic syndrome (aHUS)) who may be simultaneously exposed to more than one monoclonal antibody (e.g., anti-C5 antibody), such as when a patient is converted from treatment with one such antibody to treatment with another antibody.

### SUMMARY

Provided herein are methods of treatment designed to prevent or minimize formation of deleterious multivalent immune complexes in a human patient having a complement mediated disorder, who has been or is being treated with a first anti-C5 antibody and is then treated with a second (different) anti-C5 antibody. The disclosure is based, in part, on the discovery that large multivalent immune complexes are formed when an antigen-antibody complex (e.g., C5-eculizumab complex) is contacted (e.g., administered) with a second antibody that non-competitively associates with the complex, e.g., due to the ability of the second antibody to also bind to the antigen (e.g., C5) at an epitope that is different from the epitope of the first antibody (e.g., eculizumab). Particularly, the inventors of the instant application found that in situations where the concentration of the first antibody (e.g., eculizumab) was at or above its therapeutic dose (e.g., dose sufficient to attain minimal plasma concentration (Cmin) for C5 inhibition), administration of a second non-competitive antibody (e.g., crovalimab or pozelimab) resulted in the formation of high molecular weight (HMW) complexes, with the molecular weight (MW) of some species exceeding 1500 kDa. Surprisingly, these HMW complexes were not observed when the second antibody competed with the first antibody for the same epitope (e.g., eculizumab and ravulizumab). These data show that without a washout of the first antibody (e.g., via clearance of the antibody or inhibition of the binding of the antibody to its antigen), addition of a second non-competitive antibody results in the formation of large (e.g., hetero-oligomeric) immune complexes, including those comprising about 4 molecules of C5 bound specifically to about 5 molecules of the antibody.

In order to prevent or reduce formation of these HMW complexes with unknown PK/PD properties and safety/efficacy profiles in situations where two non-competitive antibodies are used, the instant application discloses several strategies that could be adopted to minimize risk of toxicity and/or improve safety/efficacy. One such measure involves using a weaning off period during which the first antibody is allowed to clear from the system (e.g., blood or another target tissue), prior to administration of the second antibody. Another potential strategy may involve use of apheresis procedure to remove the first antibody from the system (e.g., using a surface coated with a polypeptide comprising the antigenic determinants to which the first antibody binds). A third method may involve administering complement C5 antigen fragments containing epitopes for binding to the first antibody but not to the second antibody (in vivo or ex vivo).

The systems and methods of the instant disclosure are particularly applicable in the antibody therapy of complement-mediated disorders, e.g., PNH or aHUS, wherein the patient is switched from a first anti-C5 antibody to a second, non-competitive anti-C5 antibody. Representative examples of the first antibody include, e.g., eculizumab or ravulizumab; and representative examples of the second antibody include, e.g., 7086 antibody, 8110 antibody, 305LO5 antibody, SKY59 antibody, pozelimab, tesidolumab, crovalimab, ABP 959 antibody, ELIZARIA®, BCD-148 and/or SB12 or antigen-binding fragments thereof; preferably, 305LO5, SKY59, pozelimab, tesidolumab, crovalimab, or ABP 959.

The disclosure relates to the following non-limiting embodiments:

In some embodiments, provided herein are methods for safely treating a patient suffering from a complement mediated disorder, wherein the patient is being treated with or had been treated with a first anti-C5 antibody, the method comprising administering a therapeutically effective amount of a second anti-C5 antibody that is non-competitive with the first anti-C5 antibody after a washout sufficient to reduce levels of the first anti-C5 antibody in the patient's system such that formation of multivalent immune complexes comprising the first and the second anti-C5 antibodies and complement C5 is prevented or reduced. In some embodiments, the system comprises bodily fluid such as blood or ocular fluid. In some embodiments, the first antibody is eculizumab or ravulizumab. In some embodiments, the washout involves use of a weaning off period, e.g., a period that is equal to or greater than the half life (t^{1/2}) of the first antibody, e.g., a period≥at least 1, 2, 3 or more half-lives of the first antibody. In some embodiments, wherein the first antibody is eculizumab, the washout comprises a weaning off period of at least about 7.8 days (100 kg adult) and 19.5 days (40 kg adult), with an average weaning off period of at least about 12 days (e.g., one t^{1/2} of eculizumab). In some embodiments, wherein the first antibody is ravulizumab, the washout comprises a weaning off period of at least about 31 days (100 kg adult) to about 80 days (40 kg adult), with an average weaning off period of at least about 42 days (e.g., one t^{1/2} of ravulizumab). In some embodiments, the washout involves removal of the first antibody from the patient, e.g., using plasma apheresis. In some embodiments, the washout involves use of peptide antigens that bind to the antigen-binding regions of the first but not the second antibody. In some embodiments, the first and the second antibodies are non-competitive (e.g., do not substantially bind to the same epitope of the antigen). In some embodiments, sufficiency of the washout is determined by measuring a characteristic (e.g., mass) of the multivalent immune complex in the system. In some embodiments, the multivalent immune complex comprises at least one first antibody and at least one second antibody that are specifically bound to a single or a plurality of C5 antigens.

In one embodiment, a method for treating a patient suffering from a complement mediated disorder is provided, wherein the patient has been or is being treated with a first anti-C5 antibody, and wherein the method comprises:
(a) contacting a biological sample from the patient with a therapeutic dose of a second anti-C5 antibody under conditions sufficient for formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering to the patient whose measured level exceeds the threshold level, an adjusted regimen of the second anti-C5 antibody, wherein the adjusted regimen comprises adjustment of the dose and/or timing of administration of the second anti-C5 antibody, such that C5 inhibition is maintained, but the threshold level is not exceeded.

Also provided herein are methods for determining an adjusted regimen antibody (e.g., a regimen to prevent or minimize formation of multivalent immune complexes) comprising adjusted therapeutic dose and/or timing of administration of a second (different) anti-C5 to treat a patient suffering from a complement mediated disorder, wherein the patient has been or is being treated with a first anti-C5 antibody, the method comprising:
(a) contacting a biological sample from the patient with a therapeutic dose of the second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) adjusting the regimen of the second anti-C5 antibody therapy to the patient whose measured level exceeds the threshold level, such that C5 inhibition is maintained, but the threshold level is not exceeded.

In some embodiments, the methods further comprise weaning (e.g., withdrawing) the patient from treatment with the first anti-C5 antibody therapy.

Further provided herein are methods for switching a patient having a complement mediated disorder who has been or is being treated with a first anti-C5 antibody to treatment with a second (different) anti-C5 antibody. In one embodiment, a method for switching a patient having a complement mediated disorder who has been or is being treated with a first anti-C5 antibody to a second anti-C5 antibody is provided, the method comprising:
(a) contacting a biological sample from the patient with a therapeutic dose of a second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering, to the patient, an adjusted regimen of the second anti-C5 antibody, wherein the adjusted regimen comprises an adjustment in the dose and/or timing of administration of the second anti-C5 antibody, such that C5 inhibition is maintained, but the threshold level is not exceeded; and
(e) weaning the patient whose measured level exceeds the threshold level, from treatment with the first anti-C5 antibody therapy, thereby switching the patient from the first anti-C5 antibody to the second anti-C5 antibody.

In some embodiments of the methods described herein, the second anti-C5 antibody is administered at a reduced dose and/or a reduced frequency until the threshold is no longer exceeded.

In some embodiments of the methods described herein, the adjusted regimen comprises a modification of a clinically effective dosing or scheduling regimen. For example, in one embodiment, the adjusted regimen is a dose which is lower than a standard therapeutic dose, e.g., a sub-therapeutic dose. In other embodiments, the adjusted regimen comprises administration at a rate or interval that is moderated compared to standard scheduling, e.g., via slower rate of administration and/or less frequent administration.

Additional techniques can be used in combination with the methods described herein to clear or enhance clearance of the first anti-C5 antibody before switching to treatment with a second anti-C5 antibody. Exemplary techniques include, but are not limited to, plasmapheresis or blood transfusions.

Any suitable anti-C5 antibodies, or antigen binding fragments thereof, can be used in the methods described herein. In one embodiment, the first and/or second anti-C5 antibodies are selected from the group consisting of a full-length antibody or antigen binding fragment thereof, humanized antibody, bispecific antibody, an immunoconjugate, a chimeric antibody, a protein scaffold with antibody-like properties, such as fibronectin or ankyrin repeats, a Fab, Fab′2, scFv, affibody, avimer, nanobody, and a domain antibody. In another embodiment, the first and/or second anti-C5 antibody is a monoclonal antibody. In another embodiment, the first and second anti-C5 antibodies are antibodies that bind different epitopes on C5. In another embodiment, the first and second anti-C5 antibodies are antibodies that do not compete for binding to C5; more preferably wherein the first and/or the second anti-C5 antibodies are antibodies that bind to different domains in C5, e.g., wherein the first antibody binds to MG7 domain in C5 and the second antibody binds to MG1 or MG6 domain in C5.

An exemplary anti-C5 antibody is eculizumab. Eculizumab (also known as SOLIRIS®) is an anti-C5 antibody comprising heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. Eculizumab comprises a heavy chain variable region having the amino acid sequence set forth in SEQ ID NO: 7 and a light chain variable region having the amino acid sequence set forth in SEQ ID NO: 8. Eculizumab comprises a heavy chain comprising the amino acid sequence set forth in SEQ ID NO:10 and a light chain having the amino acid sequence set forth in SEQ ID NO:11.

In some embodiments, the antibody comprises the heavy and light chain complementarity determining regions (CDRs) or variable regions (VRs) of eculizumab. Accordingly, in one embodiment, the antibody comprises the CDR1, CDR2 and CDR3 domains of the heavy chain variable (VH) region of eculizumab having the sequence shown in SEQ ID NO:7, and the CDR1, CDR2 and CDR3 domains of the light chain variable (VL) region of eculizumab having the sequence shown in SEQ ID NO:8. In another embodiment, the antibody comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5 and 6, respectively. In another embodiment, the antibody comprises VH and VL regions having the amino acid sequences set forth in SEQ ID NO:7 and SEQ ID NO:8, respectively. In another embodiment, the antibody comprises a heavy chai comprising the amino acid sequence set forth in SEQ ID NO:10 and a light chain having the amino acid sequence set forth in SEQ ID NO:11.

Another exemplary anti-C5 antibody is ravulizumab (ULTOMIRIS®) comprising the heavy and light chains having the sequences shown in SEQ ID NOs:14 and 11, respectively, or antigen binding fragments and variants thereof. In some embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13. In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention. In another embodiment, the antibody comprises ravulizumab or a biosimilar thereof.

In some embodiments, the first and the second anti-C5 antibodies bind to different epitopes in complement C5. In some embodiments, the first anti-C5 antibody is an antibody that binds to MG7 domain in C5 and the second anti-C5 antibody is an antibody that binds to a domain other than MG7, e.g., MG1 domain or MG6 domain of C5. In some embodiments, the first anti-C5 antibody is an antibody that binds to MG1 domain in C5 and the second anti-antibody is an antibody that binds to a domain other than MG1, e.g., MG7 domain or MG6 domain of C5. In some embodiments, the first anti-C5 antibody is an antibody that binds to MG6 domain in C5 and the second anti-C5 antibody is an antibody that binds to a domain other than MG6, e.g., MG7 domain or MG1 domain of C5. In these embodiments, the anti-C5 antibody binding to MG7 domain of C5 may comprise eculizumab or ravulizumab; the antibody binding to MG1 domain may comprise crovalimab; and the antibody binding to MG6 domain may comprise pozelimab.

In another embodiment, the first and/or second anti-C5 antibody is an antibody selected from the group consisting of 7086 antibody, 8110 antibody, 305LO5 antibody, SKY59 antibody, and pozelimab (REGN3918 antibody), Tesidolumab (LFG316), Crovalimab (RG6107), ABP 959 antibody, ELIZARIA®, BCD-148 (JSC BIOCAD) and SB12 or antigen binding fragments thereof comprising, for example, the heavy and light chain CDRs of the respective antibody or a biosimilar thereof. In one embodiment, the first or, preferably, second antibody is Crovalimab, pozelimab, or a biosimilar thereof.

In another embodiment, the anti-C5 antibody comprises the heavy and light chain CDRs or variable regions of the BNJ421 antibody (described in WO2015134894 and U.S. Pat. No. 9,079,949). In another embodiment, the antibody comprises the BNJ421 antibody or a biosimilar thereof.

In another embodiment, the anti-C5 antibody comprises the heavy and light chain CDRs or variable regions of the 7086 antibody (see U.S. Pat. Nos. 8,241,628 and 8,883,158). In another embodiment, the antibody comprises the 7086 antibody or a biosimilar thereof.

In another embodiment, the anti-C5 antibody comprises the heavy and light chain CDRs or variable regions of the 8110 antibody (see U.S. Pat. Nos. 8,241,628 and 8,883,158). In another embodiment, the antibody comprises the 7086 antibody or a biosimilar thereof.

In another embodiment, the anti-C5 antibody comprises the heavy and light chain CDRs or variable regions of the 305LO5 antibody (see U.S. Pat. No. 9,765,135). In another embodiment, the antibody comprises the 7086 antibody or a biosimilar thereof.

In another embodiment, the anti-C5 antibody comprises the heavy and light chain CDRs or variable regions of the SKY59 antibody. In another embodiment, the antibody comprises the SKY59 antibody or a biosimilar thereof.

In another embodiment, the anti-C5 antibody comprises the heavy and light chain CDRs or variable regions of pozelimab (REGN3918 antibody). In another embodiment, the antibody comprises pozelimab or a biosimilar thereof.

In another embodiment, the anti-C5 antibody comprises the heavy and light chain CDRs or variable regions of tesidolumab (LFG316). In another embodiment, the antibody comprises tesidolumab or a biosimilar thereof.

In another embodiment, the anti-C5 antibody comprises the heavy and light chain CDRs or variable regions of crovalimab (RG6107). In another embodiment, the antibody comprises crovalimab or a biosimilar thereof.

In another embodiment, the anti-C5 antibody comprises the heavy and light chain CDRs or variable regions of ABP 959 antibody. In another embodiment, the antibody comprises the ABP 959 antibody or a biosimilar thereof.

In another embodiment, the anti-C5 antibody comprises the heavy and light chain CDRs or variable regions of ELIZARIA®. In another embodiment, the antibody comprises ELIZARIA® or a biosimilar thereof.

In another embodiment, the anti-C5 antibody comprises the heavy and light chain CDRs or variable regions of antibody SB12. In another embodiment, the antibody comprises the antibody SB12 or a biosimilar thereof. In another embodiment, the first anti-C5 antibody is ravulizumab (ULTOMIRIS®). In another embodiment, the first anti-C5 antibody is ravulizumab (ULTOMIRIS®) and the second antibody is 7086 antibody, 8110 antibody, 305LO5 antibody, SKY59 antibody, pozelimab (REGN3918 antibody), Tesidolumab (LFG316), Crovalimab (RG6107), ABP 959 antibody, ELIZARIA®, BCD-148 (JSC BIOCAD), SB12, antigen binding fragments thereof, or biosimilars thereof. In another embodiment, the first anti-C5 antibody is ravulizumab (ULTOMIRIS®) and the second antibody is Crovalimab, pozelimab, or a biosimilar thereof.

In another embodiment, the antibody competes for binding with, and/or binds to the same epitope on C5 as any of the above-mentioned antibodies. In another embodiment, the antibody has at least about 90% variable region amino acid sequence identity to any of the above-mentioned antibodies (e.g., at least about 90%, 95% or 99% variable region identity with SEQ ID NO:12 or SEQ ID NO:8).

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 7.4 and 25° C. with an affinity dissociation constant (K_{D}) of about 0.5 nM. In another embodiment, the antibody binds to human C5 at pH 6.0 and 25° C. with a K_{D}≥10 nM. In another embodiment, the antibody binds to human C5 at pH 6.0 and 25° C. with a K_{D} of about 22 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

In one embodiment, the threshold level is based on a minimum mass of the multivalent immune complexes. For example, in one embodiment, the threshold level is a mass of more than about 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, 750 kDa, 760 kDa, 770 kDa, 780 kDa, 790 kDa, 800 kDa, 810 kDa, 820 kDa, 830 kDa, 840 kDa, 850 kDa, 860 kDa, 870 kDa, 880 kDa, 890 kDa, 900 kDa, 910 kDa, 920 kDa, 930 kDa, 940 kDa, 950 kDa, 960 kDa, 970 kDa, 980 kDa, 990 kDa, 1000 kDa, 1010 kDa, 1020 kDa, 1030 kDa, 1040 kDa, 1050 kDa, 1060 kDa, 1070 kDa, 1080 kDa, 1090 kDa, 1100 kDa, 1110 kDa, 1120 kDa, 1130 kDa, 1140 kDa, 1150 kDa, 1160 kDa, 1170 kDa, 1180 kDa, 1190 kDa, 1200 kDa, 1210 kDa, 1220 kDa, 1230 kDa, 1240 kDa, 1250 kDa, 1260 kDa, 1270 kDa, 1280 kDa, 1290 kDa, 1300 kDa, 1310 kDa, 1320 kDa, 1330 kDa, 1340 kDa, 1350 kDa, 1360 kDa, 1370 kDa, 1380 kDa, 1390 kDa, 1400 kDa, 1410 kDa, 1420 kDa, 1430 kDa, 1440 kDa, 1450 kDa, 1460 kDa, 1470 kDa, 1480 kDa, 1490 kDa, 1500 kDa, 1510 kDa, 1520 kDa, 1530 kDa, 1540 kDa, 1550 kDa, 1560 kDa, 1570 kDa, 1580 kDa, 1590 kDa, 1500 kDa, 1610 kDa, 1620 kDa, 1630 kDa, 1640 kDa, 1650 kDa, 1660 kDa, 1670 kDa, 1680 kDa, 1690 kDa, 1700 kDa, 1710 kDa, 1720 kDa, 1730 kDa, 1740 kDa, 1750 kDa, 1760 kDa, 1770 kDa, 1780 kDa, 1790 kDa, 1800 kDa, 1810 kDa, 1820 kDa, 1830 kDa, 1840 kDa, 1850 kDa, 1860 kDa, 1870 kDa, 1880 kDa, 1890 kDa, or 1900 kDa. In another embodiment, the threshold level is a mass of more than about 532 kDa, 540 kDa, 569 kDa, 907 kDa, 913 kDa, 921 kDa, 963 kDa, 1277 kDa, 1278 kDa, 1286 kDa, 1314 kDa, 1574 kDa, 1649 kDa, 1659 kDa, or 1788 kDa.

In another embodiment, the threshold level is based on formation of multivalent immune complexes comprising more than 2 anti-C5 antibodies specifically bound to 1 molecule of complement C5 (e.g., immune complexes comprising antibody:antigen stoichiometric ratios). In another embodiment, the threshold level is formation of multivalent immune complexes consisting of more than 3 anti-C5 antibodies and 2 C5 molecules. In another embodiment, the threshold level is formation of multivalent immune complexes consisting of more than 4 anti-C5 antibodies and 3 C5 molecules. In another embodiment, the threshold level is formation of multivalent immune complexes consisting of more than 5 anti-antibodies and 4 C5 molecules.

The threshold level can be determined by any suitable assay or technique. In one embodiment, threshold level is determined by size exclusion chromatography (SEC) and/or multi-angle light scattering (MALS).

In instances where the threshold level is exceeded, administration of the second anti-antibody to the patient is deferred to allow for clearance of the first anti-C5 antibody from the patient. For example, in embodiment, administration of the second anti-C5 antibody to the patient is deferred by 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days.

The first and/or second anti-C5 antibodies can be administered according to any suitable dosing regimen. In one embodiment, the dose of the anti-C5 antibody, or antigen binding fragment thereof, is based on the weight of the patient. In certain embodiments, dosage regimens are adjusted to provide the optimum desired response (e.g., an effective response).

In one embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered at a dose of: 600 mg weekly for four weeks, followed by 900 mg for the fifth dose one week later, then 900 mg every two weeks thereafter.

In another embodiment, the patient (e.g., adult patient) has PNH and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered at a dose of: 600 mg weekly for four weeks, followed by 900 mg for the fifth dose one week later, then 900 mg every two weeks thereafter.

In another embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the patient has aHUS and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the patient has myasthenia gravis (MG) and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS® (eculizumab) and the adjusted regimen comprises an adjustment in the therapeutic regimen for therapy of the complement mediated disorder in the patient (e.g., an adult patient). In another embodiment, the therapeutic regimen comprises a dose of 600 mg weekly for four weeks, followed by 900 mg for the fifth dose one week later, then 900 mg every two weeks thereafter for the treatment of PNH. In another embodiment, the therapeutic regimen comprises a dose of 900 mg weekly for four weeks, followed by 1200 mg for the fifth dose one week later, then 1200 mg every two weeks thereafter for the treatment of aHUS. In another embodiment, the therapeutic regimen comprises a dose of 900 mg weekly for four weeks, followed by 1200 mg for the fifth dose one week later, then 1200 mg every two weeks thereafter for the treatment of MG.

In another embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., a pediatric patient) at a dose of:
(a) 900 mg weekly for four weeks, followed by 1200 mg for the fifth dose one week later, then 1200 mg every two weeks thereafter, to a patient weighing 40 kg≥,
(b) 600 mg weekly for two weeks, followed by 900 mg for the third dose one week later, then 900 mg every two weeks thereafter to a patient weighing 30 kg to <40 kg;
(c) 600 mg weekly for two weeks, followed by 600 mg for the third dose one week later, then 600 mg every two weeks thereafter, to a patient weighing 20 kg to <30;
(d) 600 mg weekly for one week, followed by 300 mg for the second dose one week later, then 300 mg every two weeks thereafter, to a patient weighing 10 kg to <20 kg; or
(e) 300 mg weekly for one week, followed by 300 mg for the second dose one week later, then 300 mg every three weeks thereafter, to a patient weighing 5 kg to <10 kg.

In another embodiment, the patient (e.g., a pediatric patient) has aHUS and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient at a dose of:
(a) 900 mg weekly for four weeks, followed by 1200 mg for the fifth dose one week later, then 1200 mg every two weeks thereafter, to a patient weighing 40 kg≥,
(b) 600 mg weekly for two weeks, followed by 900 mg for the third dose one week later, then 900 mg every two weeks thereafter to a patient weighing 30 kg to <40 kg;
(c) 600 mg weekly for two weeks, followed by 600 mg for the third dose one week later, then 600 mg every two weeks thereafter, to a patient weighing 20 kg to <30;
(d) 600 mg weekly for one week, followed by 300 mg for the second dose one week later, then 300 mg every two weeks thereafter, to a patient weighing 10 kg to <20 kg; or
(e) 300 mg weekly for one week, followed by 300 mg for the second dose one week later, then 300 mg every three weeks thereafter, to a patient weighing 5 kg to <10 kg.

In another embodiment, the second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS® (eculizumab) and the adjusted regimen comprises an adjustment in the therapeutic regimen for therapy of the complement mediated disorder in the patient (e.g., a pediatric patient). In another embodiment, the therapeutic regimen comprises administration of the second antibody to the patient at a dose of:
(a) 900 mg weekly for four weeks, followed by 1200 mg for the fifth dose one week later, then 1200 mg every two weeks thereafter, to a patient weighing 40 kg≥;
(b) 600 mg weekly for two weeks, followed by 900 mg for the third dose one week later, then 900 mg every two weeks thereafter to a patient weighing 30 kg to <40 kg;
(c) 600 mg weekly for two weeks, followed by 600 mg for the third dose one week later, then 600 mg every two weeks thereafter, to a patient weighing 20 kg to <30;
(d) 600 mg weekly for one week, followed by 300 mg for the second dose one week later, then 300 mg every two weeks thereafter, to a patient weighing 10 kg to <20 kg; or
(e) 300 mg weekly for one week, followed by 300 mg for the second dose one week later, then 300 mg every three weeks thereafter, to a patient weighing 5 kg to <10 kg.

In another embodiment, the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO: 8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient (e.g., an adult patient) (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the patient (e.g., adult patient) has PNH of aHUS and the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), and the adjusted regimen comprises an adjustment in the therapeutic regimen for therapy of the complement mediated disorder in the patient (e.g., an adult patient). In another embodiment, the therapeutic regimen comprises administration of the second antibody to the patient at a dose of: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO: 8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient (e.g., a pediatric patient): (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the patient (e.g., pediatric patient) has PNH of aHUS and the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), and the adjusted regimen comprises an adjustment in the therapeutic regimen for therapy of the complement mediated disorder in the patient (e.g., pediatric patient). In another embodiment, the therapeutic regimen comprises administration of the second antibody to the patient (e.g., pediatric patient) at a dose of: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

The first and/or second anti-C5 antibody can be administered to the patient via any suitable means or art recognized technique. In one embodiment, the first and/or second anti-C5 antibody is administered intravenously to the patient. In another embodiment, the first and/or second anti-C5 antibody is administered subcutaneously to the patient.

Any biological sample can be used in the treatment methods described herein. Exemplary biological samples include but are not limited to blood, serum, plasma, urine, saliva, lymph, spinal fluid, intercellular fluid, vitreous humor, and sweat. In one embodiment, the biological fluid is blood.

In one embodiment, the biological sample from the patient is contacted with the therapeutic dose of a second anti-C5 antibody in vivo. In one embodiment, the biological sample from the patient is contacted with the therapeutic dose of a second anti-C5 antibody ex vivo. In another embodiment, the biological sample from the patient is contacted with the therapeutic dose of a second anti-C5 antibody in vitro.

Patients treated according to the methods described herein have one or more complement mediated disorders. Exemplary complement mediated disorders include, but are not limited to rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome. In a one embodiment, the complement mediated disorder is aHUS. In another embodiment, the complement mediated disorder is PNH.

The efficacy of the treatment methods provided herein can be assessed using any suitable means. In one embodiment, the treatment resolves at least one sign or symptom of the complement-mediated disorder without toxicity associated with multivalent immune complexes formed as a result of administration of the second anti-C5 antibody.

In some embodiments, the disclosure relates to use of a therapeutically effective amount of a second anti-C5 antibody that is non-competitive with a first anti-C5 antibody, for the treatment of a complement-mediated disease (e.g., aHUS, PNH, HSCT-TMA, CM-TMA, NMOSD, gMG or ALS) in a patient who is being treated or has been treated with the first anti-C5 antibody, wherein the second anti-C5 antibody is administered after a washout sufficient to reduce levels of the first anti-C5 antibody in the patient's system (e.g., blood) such that formation of multivalent immune complexes comprising the first and the second anti-C5 antibodies and complement C5 is prevented or reduced. Preferably, the multivalent immune complex comprises at least one first antibody and at least one second antibody that are specifically bound to a complement C5 antigen, and optionally, additional C5 antigens.

In some embodiments, the disclosure relates to a use, according to the foregoing, of a therapeutically effective amount of a second antibody that is non-competitive with a first antibody selected from eculizumab and ravulizumab or a biosimilar of the first antibody.

In some embodiments, the disclosure relates to a use, according to the foregoing, of a therapeutically effective amount of a second antibody that is non-competitive with eculizumab or ravulizumab, wherein the second antibody is selected from 305LO5, SKY59, pozelimab, tesidolumab, crovalimab, or ABP 959 or a biosimilar thereof; preferably, wherein the second antibody is pozelimab, tesidolumab, or crovalimab.

### BRIEF DESCRIPTION OF THE DRAWINGS

FIGS. 1A-1D show that recombinant anti-C5 monoclonal antibodies crovalimab mimetic and pozelimab mimetic bind to human C5 non-competitively with eculizumab. Data are shown for the following different second antibodies: mAb N19/8 which is known to bind to a different site on C5 to eculizumab (FIG. 1A), ravulizumab (FIG. 1B), mAb crovalimab mimetic (FIG. 1C), and mAb pozelimab mimetic (FIG. 1D). The scale bar shows the deflection corresponding to a biosensor signal of 1.0 nm.

FIGS. 2A-2D show that crovalimab mimetic induces formation of very large complexes in the presence of eculizumab and C5 at eculizumab and C5 concentrations mimicking those in human blood as eculizumab approaches Cₘᵢₙ. As shown in FIG. 2A, crovalimab mimetic (700 nM) alone migrated as a single peak and formed two additional species in the presence of C5 (400 nM) corresponding to 1:1 and 1:2 crovalimab mimetic:C5 complexes. As shown in FIG. 2B, reconstitution of crovalimab mimetic (700 nM) with eculizumab (700 nM) plus C5 (400 nM) resulted in the formation of a series of larger, earlier eluting peaks denoted with asterisks. As shown in FIGS. 2C and 2D, complexes formed between crovalimab mimetic, C5 and eculizumab in PBS were in some cases greater than 1.5 million Da by MALS irrespective of whether reconstitution was with a concentration of crovalimab mimetic equimolar (700 nM) to eculizumab (FIG. 2C) or fivefold molar excess (3500 nM) of eculizumab (FIG. 2D).

FIGS. 3A-3D show that pozelimab mimetic induces formation of very large complexes in the presence of eculizumab plus C5 at concentrations of eculizumab and C5 mimicking those in human blood as eculizumab approaches Cₘᵢₙ. As shown in FIG. 3A, pozelimab mimetic (700 nM) alone migrated as a single peak and formed two additional species in the presence of C5 (400 nM) corresponding to 1:1 and 1:2 pozelimab mimetic:C5 complexes. Reconstitution of pozelimab mimetic (700 nM) with eculizumab (700 nM) plus C5 (400 nM) resulted in the formation of a series of larger, earlier eluting peaks denoted with asterisks. As shown in FIG. 3B, earlier eluting complexes with highly similar retention times (*) were also observed when pozelimab mimetic (700 nM) was reconstituted with labeled eculizumab (700 nM) in 80% human plasma, but not when pozelimab mimetic was absent. As shown in FIGS. 3C-3D, complexes formed between pozelimab mimetic, C5 and eculizumab in PBS were in some cases greater than 1.5 million Da by MALS (FIG. 3C) and remained large even when pozelimab mimetic was at 3500 nM, a fivefold excess to eculizumab (FIG. 3D).

FIGS. 4A-4D show that ravulizumab forms only 1:1 and 1:2 complexes with C5 both in the absence or presence of eculizumab. As shown in FIG. 4A, ravulizumab alone migrated as a single peak, but in the presence of C5 or C5 plus eculizumab three peaks were observed. As shown in FIG. 4B, the last peak to elute in the composition of C5 plus eculizumab and ravulizumab corresponds to unbound antibody and had a shoulder because free eculizumab and ravulizumab had slightly different retention times. The compositions comprising ravulizumab and C5 (FIG. 4C), and ravulizumab and C5 plus eculizumab (FIG. 4D) exhibited masses by MALS consistent with the formation of only 1:1 and 1:2 mAb:C5 complexes.

FIG. 5 shows that masses of the complexes formed between eculizumab, C5 and either of the anti-C5 mAbs, crovalimab mimetic or pozelimab mimetic are consistent with formation of complexes containing multiple antibody and C5 molecules.

### DETAILED DESCRIPTION

### I. General Definitions

The term “and/or” includes any and all possible combinations of one or more of the associated listed items, as well as the lack of combinations when interpreted in the alternative (“or”).

The term “about” means a range of plus or minus 10% of that value, e.g., “about 5” means 4.5 to 5.5, unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5.

Where a range of values is provided in this disclosure, it is intended that each intervening value between the upper and lower limit of that range and any other stated or intervening value in that stated range is encompassed within the disclosure. For example, if a range of 1 mM to 8 mM is stated, it is intended that 2 mM, 3 mM, 4 mM, 5 mM, 6 mM, and 7 mM are also explicitly disclosed.

As used herein, the term “plurality” can be 2, 3, 4, 5, 6, 7, 8, 9, 10, or more.

The terms “first” and “second” are not to be construed as having a relation in time or space but merely relate to distinction therebetween, e.g., in structure, property, or function.

The term “substantially” means sufficient to work for the intended purpose. The term “substantially” thus allows for minor, insignificant variations from an absolute or perfect state, dimension, measurement, result, or the like such as would be expected in the field that do not appreciably affect overall performance (e.g., +/−10%).

As used herein, the term “marker” refers to a characteristic that can be objectively measured as an indicator of normal biological processes, pathogenic processes or a pharmacological response to a therapeutic intervention, e.g., treatment with anti-C5 antibody.

As used herein, a “panel” refers to a group of two or more distinct molecular species that have shown to be indicative of or otherwise correlated with a particular disease or health condition. Such “molecular species” may be referred to as “biomarkers”, with the term “biomarker” being understood to mean a biological molecule, the presence or absence or level of which serves as an indicator of a particular biological state, for example, the occurrence (or likelihood) of toxicity due to build-up of immune complexes in a subject. In other words, a biomarker is a characteristic that can objectively-measured and evaluated as an indicator of normal biologic processes, pathogenic processes, or pharmacologic responses to a drug.

The term “modulate” refers to enhancement (e.g., an increase) or inhibition (e.g., a decrease) in the specified level or activity. The term “enhance” or “increase” refers to an increase in the specified parameter (e.g., mass of immune complexes) of at least about 1.25-fold, 1.5-fold, 2-fold, 3-fold, 4-fold, 5-fold, 6-fold, 8-fold, 10-fold, twelvefold, or even fifteen-fold. The term “inhibit” or “reduce” or grammatical variations thereof refers to a decrease or diminishment in the specified level or activity of the target, e.g., little or essentially no detectible level or activity of the target (at most, an insignificant amount).

As used herein the term “reducing” refers to a decrease or lessening by an appreciable, e.g., statistically significant, amount or quality. In embodiments, reducing refers to either partially or completely inhibiting an activity or decreasing or lowering an activity. In other embodiments, “reducing” means a decrease by at least 10% compared to a reference level, for example a decrease by at least about 15%, 20%, 25%, 30%, 35%, 40%, 45%, 50%, 55%, 60%, 65%, 70%, 75%, 80%, 85%, 90%, 95%, or up to and including a 100% decrease compared to a reference level. As used herein, the term “reducing the incidence” and “improving function” refer to a beneficial effect, e.g., amelioration or an improvement over baseline. Frequently the improvement over baseline is statistically significant. For example, “reducing the incidence” and “improving function” may refer to an amelioration of at least about 10%, 20%, 30%, 40%, 50%, 60%, 70%, 80%, 90%, or 100%, as compared to a reference level, or at least about a 1.5-fold, 2-fold, 3-fold, 4-fold, 5-fold, 6-fold, 7-fold, 8-fold, 9-fold, 10-fold, or more, e.g., 20-fold, as compared to a control.

As used herein, the term “diagnosis” refers to methods by which a determination can be made as to whether a subject is likely to be suffering from a given disease or condition, e.g., complement-mediated diseases. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., unexplained changes in vitals, or phenotypic, genotypic or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given trait.

As used herein, the term “detecting,” refers to the process of determining a value or set of values associated with a sample by measurement of one or more parameters in a sample and may further comprise comparing a test sample against reference sample. For example, the detection of antibody-mediated rejection may include identifying, assaying, measuring and/or quantifying a marker, e.g., formation of multivalent immune complexes.

As used herein the term “determining” refers to the process of scientifically measuring a phenomenon as it relates to a process, e.g., formation of immune complexes in vivo. It assumes one skilled in the art will be making tests within that skill to observe and measure the phenomenon. The term “monitor” refers to determining the progression of the condition or determining the effectiveness of a particular treatment protocol (e.g., weaning off antibody therapy) or a composition (e.g., administration of neutralizing peptides).

The term “likelihood,” as used herein, generally refers to a probability, a relative probability, a presence or an absence, or a degree. As used herein, the term “at risk” for a disease or disorder refers to a subject (e.g., a human) that is predisposed to experiencing a particular disease. This predisposition may be genetic or environmental. Thus, it is not intended that the present disclosure be limited to any particular risk. The term “outcome” means a specific result or effect of diagnosis or therapy that can be measured.

The term “control,” as used herein, refers to a reference for a test sample, such as control healthy subjects or untreated subjects, and the like. A “reference sample,” as used herein, refers to a sample of tissue or cells that may or may not have a disease that are used for comparisons. Thus a “reference” sample may provide a basis to which another sample, for example, a sample of a patient who was treated in accordance with the present disclosure, can be compared. In contrast, a “test sample” refers to a sample compared to a reference sample. The reference sample need not be disease free, such as when reference and test samples are obtained from the same patient who were treated in accordance with the present methods (e.g., incorporating a washout before administration of the second antibody) or not (e.g., not incorporating the washout).

As used herein, the term “threshold” refers to a parameter (e.g., level or amount) of the marker (e.g., multivalent immune complex) in a particular setting (e.g., in a sample from a subject that has received a monotherapy of anti-C5 antibody).

The term “level” can refer to binary (e.g., absent/present), qualitative (e.g., absent/low/medium/high), or quantitative information (e.g., a value proportional to number, frequency, or concentration) indicating the presence of a particular molecular species.

As used herein, the term “subject” or “patient” is a human patient (e.g., a patient having a complement mediated disorder, such as PNH or aHUS).

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (≥18 years of age). As used herein, a subject “in need of prevention,” “in need of treatment,” or “in need thereof,” refers to one, who by the judgment of an appropriate medical practitioner (e.g., a doctor, a nurse, or a nurse practitioner in the case of humans), would reasonably benefit from a given treatment, e.g., a particular therapeutic or prophylactic agent to treat a condition.

As used herein, the term “treat” or “treating” refers to providing an intervention, e.g., providing any type of medical or surgical management of a subject. The treatment can be provided to reverse, alleviate, inhibit the progression of, prevent or reduce the likelihood of a disorder or condition, or to reverse, alleviate, inhibit or prevent the progression of, prevent or reduce the likelihood of one or more symptoms or manifestations of a disorder or condition. “Prevent” refers to causing a disorder or condition, or symptom or manifestation of such not to occur for at least a period of time in at least some individuals. Treating can include administering a therapeutic agent (e.g., an anti-C5 antibody) to the subject following the development of one or more symptoms or manifestations indicative of condition, e.g., to reverse, alleviate, reduce the severity of, and/or inhibit or prevent the progression of the condition and/or to reverse, alleviate, reduce the severity of, and/or inhibit or one or more symptoms or manifestations of the condition. Accordingly, a composition can be administered to a subject who has developed a complement-mediated disorder or is at increased risk of developing the complement-mediated disorder relative to a member of the general population. Such a composition can be administered prophylactically, e.g., before development of any symptom or manifestation of the condition. Preferably, the composition is administered therapeutically, e.g., after development of any symptom or manifestation of the condition.

The term “symptom” refers to an indication of disease, illness, injury, or that something is not right in the body. Symptoms are felt or noticed by the individual experiencing the symptom, but may not easily be noticed by others. The term “sign” refers an indication that something is not right in the body, which can be seen by a doctor, nurse, or other health care professional.

The term “administration” or “administering” when used in conjunction with an agent, e.g., drug, means to deliver the agent directly into or onto a cell or target tissue or to provide the agent to a patient whereby it impacts the tissue to which it is targeted. The term “contact” refers to bringing an agent (e.g., anti-C5 antibody) and the target (e.g., C5) in sufficiently close proximity to each other for one to exert a biological effect on the other (e.g., inhibition of the target). In some embodiments, the term contact means binding of the agent to the target.

As used herein, “transfusion” refers to an act of transferring blood, blood products, or other fluid, e.g., saline, into the circulatory system of a subject.

The term “intravenous” generally means “within a vein” and refers to accessing a subject's target cells or tissue via the vasculature system. In intravenous (IV) therapy, liquid substances are administered directly into a vein. Compared with other routes of administration, the intravenous route is probably the fastest way to deliver agents throughout a body. Some medications, blood transfusions, and parenteral (e.g., non-alimentary) nutrients are administered intravenously using standard delivery systems.

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one sign/symptom of a complement-mediated disorder (e.g., PNH, aHUS, HSCT-TMA, CM-TMA, NMOSD, gMG or ALS). In embodiments, effective treatment may refer to alleviation of at least one sign/symptom of PNH (e.g., pallor, fatigue, jaundice, anemia, cytopenia, abdominal pain, dyspnea, dysphagia, chest pain or erectile dysfunction). In embodiments, effective treatment may refer to alleviation of at least one sign/symptom of aHUS (e.g., severe hypertension, proteinuria, uremia, lethargy/fatigue, irritability, thrombocytopenia, microangiopathic hemolytic anemia, and renal function impairment (e.g., acute renal failure)). In embodiments, effective treatment may refer to alleviation of at least one sign/symptom of hematopoietic stem cell transplantation (HSCT)-TMA, e.g., TMA after HSCT (e.g., elevated serum creatinine, reduced glomerular filtration rate (GFR); hypertension requiring medication(s); and elevated proteinuria or urine protein creatinine ratio). In embodiments, effective treatment may refer to alleviation of at least one sign/symptom of complement-mediated TMA (CM-TMA), e.g., abdominal pain, confusion, fatigue, edema, nausea, vomiting and diarrhea. In embodiments, effective treatment may refer to alleviation of at least one sign/symptom of NMO selected from loss of vision and spinal cord function (e.g., decreased visual acuity, possibly with loss of color vision; blindness in one or both eyes in five years; overall muscle weakness and reduced sensation; and loss of bladder and bowel control). In embodiments, effective treatment may refer to alleviation of at least one sign/symptom of generalized myasthenia gravis (gMG), e.g., ocular weakness, causing ptosis (drooping eyelids) and/or diplopia (double vision), leg weakness, dysphagia and slurred or nasal speech, especially wherein the symptoms worsen with various stressors, such as, e.g., exertion, heat and infection. In embodiments, effective treatment may refer to alleviation of at least one sign/symptom of amyotrophic lateral sclerosis (ALS) (e.g., stiff muscles, muscle weakness, muscle wasting, difficulty speaking, difficulty swallowing, difficulty breathing, difficulty chewing, difficulty walking, fasciculations, cramps, or any combination thereof).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one sign/symptom of the complement-mediated disorder (e.g., PNH, aHUS, HSCT-TMA, CM-TMA, NMOSD, gMG or ALS), as provided above. An effective amount can be administered in one or more administrations.

As used herein, the term “loading dose” refers to the first dose administered (e.g., during an administration cycle). As used herein, the terms “maintenance” and “maintenance phase” are used interchangeably and refer to the second phase of treatment. In certain embodiments, treatment is continued as long as clinical benefit is observed or until unmanageable toxicity or disease progression occurs. As used herein, “therapeutic dose” means any suitable amount or concentration of the drug to be administered to a subject as part of a prescribed regimen that is effective for treating a complement-mediated disorder. The specific dosage is a matter of design choice and may vary with the traits of the subject.

As used herein, the term “sub-therapeutic” refers to an amount/dose of a drug (e.g., anti-C5 antibody) that is below the amount of the drug that is conventionally used to treat a complement-mediated disease. For example, a sub-therapeutic amount is an amount less than that defined by the manufacturer as being required or recommended for therapy of a disorder.

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

The term “inhibitor” or “antagonist” as used herein refers to a substance that interferes with the effects of another substance. Functional or physiological antagonism occurs when two substances produce opposite effects on the same physiological function. Chemical antagonism or inactivation is a reaction between two substances to neutralize their effects, e.g., binding of an antibody to an antigen, which prevents the antigen from acting on its target. Dispositional antagonism is the alteration of the disposition of a substance (its absorption, biotransformation, distribution, or excretion) so that less of the agent reaches the target or its persistence there is reduced.

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, AFFIBODY®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

Antibodies that are “competitive” “compete” with another antibody for binding to a target refer to antibodies that inhibit (partially or completely) the binding of the other antibody to the target. In some embodiments, competitive antibodies are capable of displacing each other from the antigen-antibody complex. In contrast, antibodies that are “non-competitive” do not compete with another antibody for binding to the target, i.e., do not inhibit (partially or completely) the binding of the other antibody to the target. In some embodiments, non-competitive antibodies are incapable of displacing each other from the complex.

The term “biosimilar” refers to a biological product that is highly similar, e.g., in primary structure, such as amino acid sequence, to a licensed reference biological product. As a representative example, eculizumab biosimilars include, e.g., ABP 959 (Amgen); Elizaria (Generium); and SB12 (Samsung).

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method.

As used herein, the term “C5 inhibition” relates to inhibition or antagonism of complement C5 in the complement pathway. Suitable methods for measuring inhibition of C5 cleavage are known in the art. For example, the concentration and/or physiologic activity of C5a and/or C5b in a body fluid can be measured, e.g., chemotaxis assays, IAs, or ELISAs (for C5a); and hemolytic assays or assays for soluble C5b-9 (for C5b). Other assays for measuring TCC formation, inhibition of complement component C5, which modulates cell lysis, can be measured using a conventional hemolytic assay.

As used herein, the term “washout” refers to, in the broadest sense, a reduction in the level (e.g., amount or concentration) or activity (e.g., neutralizing property) of a composition (e.g., a drug such as an anti-C5 antibody) from a system (e.g., blood).

As used herein, the term “level” refers to, in the broadest sense, a measure of the physical property, such as amount (mass/weight) or concentration (amount/volume) and also non-physical properties (e.g., activity as measured by enzyme kinetics, signaling activity).

As used herein, the term “multivalent immune complex” refers to an antigen-antibody complex that is formed, e.g., via recognition of a plurality of epitopes in a single antigen by a plurality of different antibodies that hind specifically thereto. Representative examples include antibody-antigen complexes indicating antibody:antigen stochiometric ratios that are greater than 2:1, e.g., 3:2, 4:3, 5:4, and more. Specific types of multivalent immune complexes comprising C5-αC5 antibodies, as determined by mass ratios, are provided in the Examples.

The term “half-life” as used here in relation to a biological (such as an antibody) generally refers to the time taken for the concentration of the biological in the system (e.g., blood or similar bodily fluid) to be reduced by 50%, for example due to degradation of the biological and/or clearance or sequestration of the biological by natural mechanisms. In the context of in vivo half-life, this parameter can be determined in any manner known per se, such as by pharmacokinetic (PK) analysis. Suitable techniques are described in U.S. Pat. No. 8,629,244 (and equivalent WO 2008/020079). Typically, the half-life can be expressed using parameters such as the t^{1/2}-alpha, t^{1/2}-beta and the area under the curve (AUC). Partly due to physiological factors, in vivo half-life of an antibody depends on the weight of the subject.

The term “apheresis” encompasses a medical technology in which the blood of a subject (e.g., patient) is passed through an apparatus that separates out one particular constituent or component and returns the remainder to the circulation. It is thus an extracorporeal therapy. If plasma is separated, then the apheresis is called plasmapheresis. In antigen-specific antibody apheresis, antigen-specific antibodies are removed from the plasma from the patients by using antigen-specific determinants (e.g., peptides containing epitopes that are specific for a target antibody, e.g., C5 antibody) and returning the other remaining components of plasma to the same patients. However, plasmapheresis or selective apheresis, which nonspecifically removes total immunoglobulins from plasma, may also be used.

The term “cell” refers to basic building blocks of tissue, such as cells from a human. Included are normal cells and transformed cells.

### II. Anti-C5 Antibodies

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, Affibody®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

Anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises ravulizumab or a biosimilar thereof.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises antibody BNJ421 or a biosimilar thereof.

The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs:21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28. In another embodiment, the antibody comprises ravulizumab or a biosimilar thereof. In another embodiment, the antibody comprises 7086 antibody or a biosimilar thereof.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36. In another embodiment, the antibody comprises 8110 antibody or a biosimilar thereof.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs:37, 38 and 39, respectively, and light chain CDR1, CDR2 and CDR3 domains having the sequences set forth in SEQ ID NOs:40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44. In another embodiment, the antibody comprises 305LO5 antibody or a biosimilar thereof.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody. In another embodiment, the antibody, or antigen binding fragment thereof, comprises a heavy chain comprising SEQ ID NO:45 and a light chain comprising SEQ ID NO:46. In another embodiment, the antibody comprises SKY59 antibody or a biosimilar thereof.

In some embodiments, the anti-C5 antibody comprises the heavy and light chain variable regions or heavy and light chains of the pozelimab (REGN3918 antibody; see U.S. Pat. No. 10,633,434). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50. In another embodiment, the antibody comprises pozelimab or a biosimilar thereof.

In other embodiments, the anti-C5 antibody comprises the heavy and light chain CDRs or variable regions of tesidolumab (LFG316). In another embodiment, the antibody comprises tesidolumab or a biosimilar thereof.

In another embodiment, the anti-C5 antibody comprises the heavy and light chain CDRs or variable regions of crovalimab (RG6107). In another embodiment, the antibody comprises crovalimab or a biosimilar thereof.

In another embodiment, the anti-C5 antibody comprises the heavy and light chain CDRs or variable regions of ABP 959 antibody. In another embodiment, the antibody comprises ABP 959 or a biosimilar thereof.

In another embodiment, the anti-C5 antibody comprises the heavy and light chain CDRs or variable regions of ELIZARIA®. In another embodiment, the antibody comprises ELIZARIA® or a biosimilar thereof.

In another embodiment, the anti-C5 antibody comprises the heavy and light chain CDRs or variable regions of antibody SB12. In another embodiment, the antibody comprises antibody SB12 or a biosimilar thereof.

In another embodiment, the first anti-C5 antibody is ravulizumab (ULTOMIRIS®). In another embodiment, the first anti-C5 antibody is ravulizumab (ULTOMIRIS®) and the second antibody is 7086 antibody, 8110 antibody, 305LO5 antibody, SKY59 antibody, pozelimab (REGN3918 antibody), Tesidolumab (LFG316), Crovalimab (RG6107), ABP 959 antibody, ELIZARIA®, BCD-148 (JSC BIOCAD), SB12, antigen binding fragments thereof, or biosimilars thereof. In another embodiment, the first anti-C5 antibody is ravulizumab (ULTOMIRIS®) and the second antibody is Crovalimab, pozelimab, or a biosimilar thereof.

In some embodiments, the first anti-C5 antibody comprises eculizumab (SOLIRIS®) or ravulizumab (ULTOMIRIS®) or an antigen-binding fragment thereof (e.g., comprising heavy and light chain complementarity determining regions (HCDR1-3 and LCDR1-3, respectively) of eculizumab) and the second anti-C5 antibody is not a biosimilar of eculizumab (SOLIRIS®), e.g., is not an antibody selected from ABP 959 antibody (manufactured by Amgen Inc., USA), ELIZARIA® (manufactured by Generium JNC, Russia), or SB12 (manufactured by Samsung Bioepis, Incheon, South Korea).

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAE VKKPGASVKV SCKASGHIFS NYWIQWVRQA
PGQGLEWMGE ILPGSGHTEY TENFKDRVTM TRDTSTSTVY
MELSSLRSED TAVYYCARYF FGSSPNWYFD VWGQGTLVTV
SS.

In some embodiments, an anti-C5 antibody described herein comprises a light chain variable region comprising the following amino acid sequence:

(SEQ ID NO: 8)
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J. Biol. Chem., 282:1709-17, 2007), the disclosures of each of which are incorporated herein by reference in their entirety.

In some embodiments, the variant constant region has a substitution at EU amino acid position 255 for valine. In some embodiments, the variant constant region has a substitution at EU amino acid position 309 for asparagine. In some embodiments, the variant constant region has a substitution at EU amino acid position 312 for isoleucine. In some embodiments, the variant constant region has a substitution at EU amino acid position 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO:11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25° C. (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or nM. In some embodiments, the antibody binds to C5 at pH 6.0 and 25° C. (and, otherwise, under physiologic conditions) with a K_{D} that is about 22 nM.

In other embodiments, the [(K_{D} of the antibody for C5 at pH 6.0 at 25° C.)/(K_{D} of the antibody for C5 at pH 7.4 at 25 C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000)

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J. Immunol. Meth., 160:191-8, 1993; Jonsson, U. et al., Ann. Biol. Clin., 51:19-26, 1993; Jonsson, U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations can be measured, for example, at 25 C or 37 C (see the working examples). The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50 eq assay.

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen:antibody complexes, and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

In some embodiments, the first and the second anti-C5 antibodies of the disclosure bind to different domains in complement C5. The various domains in C5 include, e.g., M1 domain, M6 domain and M7 domain. Representative examples of anti-C5 antibodies binding to MG7 domain of C5 include, e.g., eculizumab and ravulizumab; representative examples of anti-C5 antibodies binding to MG1 domain of C5 include, e.g., crovalimab; and representative examples of anti-C5 antibodies binding to MG6 domain of C5 include, e.g., pozelimab. See Schatz-Jakobsen et al. (J Immunol., 2016 Jul. 1; 197(1):337-44); Fukuzawa et al. (Sci Rep., 2017 Apr. 24; 7(1):1080); and Latuszek et al. (PLoS One, 2020 May 8; 15(5):e0231892). In some embodiments, the first anti-C5 antibody is an antibody that binds to MG7 domain in C5 and the second anti-C5 antibody is an antibody that binds to a domain other than MG7, e.g., MG1 domain or MG6 domain of C5. In some embodiments, the first anti-C5 antibody is an antibody that binds to MG1 domain in C5 and the second anti-C5 antibody is an antibody that binds to a domain other than MG1, e.g., MG7 domain or MG6 domain of C5. In some embodiments, the first anti-C5 antibody is an antibody that binds to MG6 domain in C5 and the second anti-C5 antibody is an antibody that binds to a domain other than MG6, e.g., MG7 domain or MG1 domain of C5.

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Kohler, G. & Milstein, C., Eur. J. Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

The first and/or second anti-C5 antibody can be administered to the patient via any suitable means or art recognized technique. In one embodiment, the first and/or second anti-antibody is administered intravenously to the patient. In another embodiment, the first and/or second anti-C5 antibody is administered subcutaneously to the patient.

### III. Multivalent Immune Complexes

When more than one antibody binds to a monovalent target antigen at different sites, multivalent immune complexes can form. As used herein, the phrase “multivalent immune complex” refers to an immune complex formed when more than two antibody molecules (e.g., anti-C5 antibody molecules) bind specifically to a target antigen (e.g., a molecule of complement C5). The valency of such complexes can affect clearance, effector function, uptake by phagocytic cells, and have deleterious effects. Accordingly, the methods described herein are designed to prevent or minimize formation of such multivalent immune complexes in a patient who has been or is being treated with a first anti-C5 antibody and is then treated with a second anti-C5 antibody.

In one embodiment, the methods described herein include a step of contacting a biological sample (e.g., blood, serum, plasma, urine, saliva, lymph, spinal fluid, intercellular fluid, vitreous humor, or sweat) from the patient with a therapeutic dose of a second anti-C5 antibody under conditions sufficient for formation of multivalent immune complexes comprising complement C5, the first anti-C5 antibody, and second anti-C5 antibody. In one embodiment, the contacting step occurs in vivo. In a preferable embodiment, the contact step occurs ex vivo or in vitro.

In another embodiment, the methods further include a step of measuring a level of the multivalent immune complexes formed in the first contacting step. Levels of multivalent immune complexes can be determined by any suitable assay or technique. In one embodiment, the level is determined by size exclusion chromatography (SEC). SEC, also known as molecular sieve chromatography, is a chromatographic method in which molecules in solution are separated by their size, and in some cases molecular weight. It is usually applied to large molecules or macromolecular complexes such as proteins and industrial polymers. In another embodiment, the level is determined by multi-angle light scattering (MALS). MALS is a technique for measuring the light scattered by a sample into a plurality of angles. It is used for determining both the absolute molar mass and the average size of molecules in solution, by detecting how they scatter light. In another embodiment, the level is determined by SEC and MALS.

In another embodiment, the methods further include a step of determining if the measured level of multivalent immune complexes exceeds a threshold level. In one embodiment, the threshold level is based on a minimum mass of the multivalent immune complexes. For example, in one embodiment, the threshold level is a mass of more than about 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, 750 kDa, 760 kDa, 770 kDa, 780 kDa, 790 kDa, 800 kDa, 810 kDa, 820 kDa, 830 kDa, 840 kDa, 850 kDa, 860 kDa, 870 kDa, 880 kDa, 890 kDa, 900 kDa, 910 kDa, 920 kDa, 930 kDa, 940 kDa, 950 kDa, 960 kDa, 970 kDa, 980 kDa, 990 kDa, 1000 kDa, 1010 kDa, 1020 kDa, 1030 kDa, 1040 kDa, 1050 kDa, 1060 kDa, 1070 kDa, 1080 kDa, 1090 kDa, 1100 kDa, 1110 kDa, 1120 kDa, 1130 kDa, 1140 kDa, 1150 kDa, 1160 kDa, 1170 kDa, 1180 kDa, 1190 kDa, 1200 kDa, 1210 kDa, 1220 kDa, 1230 kDa, 1240 kDa, 1250 kDa, 1260 kDa, 1270 kDa, 1280 kDa, 1290 kDa, 1300 kDa, 1310 kDa, 1320 kDa, 1330 kDa, 1340 kDa, 1350 kDa, 1360 kDa, 1370 kDa, 1380 kDa, 1390 kDa, 1400 kDa, 1410 kDa, 1420 kDa, 1430 kDa, 1440 kDa, 1450 kDa, 1460 kDa, 1470 kDa, 1480 kDa, 1490 kDa, 1500 kDa, 1510 kDa, 1520 kDa, 1530 kDa, 1540 kDa, 1550 kDa, 1560 kDa, 1570 kDa, 1580 kDa, 1590 kDa, 1500 kDa, 1610 kDa, 1620 kDa, 1630 kDa, 1640 kDa, 1650 kDa, 1660 kDa, 1670 kDa, 1680 kDa, 1690 kDa, 1700 kDa, 1710 kDa, 1720 kDa, 1730 kDa, 1740 kDa, 1750 kDa, 1760 kDa, 1770 kDa, 1780 kDa, 1790 kDa, 1800 kDa, 1810 kDa, 1820 kDa, 1830 kDa, 1840 kDa, 1850 kDa, 1860 kDa, 1870 kDa, 1880 kDa, 1890 kDa, or 1900 kDa. In another embodiment, the threshold level is a mass of more than about 532 kDa, 540 kDa, 569 kDa, 907 kDa, 913 kDa, 921 kDa, 963 kDa, 1277 kDa, 1278 kDa, 1286 kDa, 1314 kDa, 1574 kDa, 1649 kDa, 1659 kDa, or 1788 kDa.

In another embodiment, the threshold level is based on formation of multivalent immune complexes comprising more than 2 anti-C5 antibodies specifically bound to 1 molecule of complement C5 (e.g., immune complexes comprising antibody:antigen stoichiometric ratios). In another embodiment, the threshold level is formation of multivalent immune complexes consisting of more than 3 anti-C5 antibodies and 2 C5 molecules. In another embodiment, the threshold level is formation of multivalent immune complexes consisting of more than 4 anti-C5 antibodies and 3 C5 molecules. In another embodiment, the threshold level is formation of multivalent immune complexes consisting of more than 5 anti-antibodies and 4 C5 molecules.

### IV. Complement Mediated Disorders

Provided herein are methods of involving a human patient having a complement mediated disorder, who has been or is being treated with a first anti-C5 antibody and is then treated with a second anti-C5 antibody. Patients treated according to the methods described herein have one or more complement mediated disorders. Exemplary complement mediated disorders include, but are not limited to rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome. In a one embodiment, the complement mediated disorder is aHUS. In another embodiment, the complement mediated disorder is PNH.

### V. Therapeutic Regimens and Adjusted Regimens

In the methods described herein, the patient has been or is being treated with a first anti-C5 antibody and is then is treated with (e.g., switched to) a second (different) anti-C5 antibody. The first and/or second anti-C5 antibodies can be administered according to any suitable therapeutic regimen. In some embodiments, the first and/or second anti-C5 antibodies are administered according to a clinically effective dosing or scheduling regimen.

In other embodiments of the methods described herein, the therapeutic regimen is adjusted, e.g., to prevent or minimize multivalent immune complex formation when the patient is transitioned from the first anti-C5 antibody to the second anti-C5 antibody. The adjusted regimen comprises a modification of a clinically effective dosing or scheduling regimen. For example, in one embodiment, the adjusted regimen is a dose which is lower than a standard therapeutic dose, e.g., a sub-therapeutic dose. In other embodiments, the adjusted regimen comprises administration at a rate or interval that is moderated compared to standard scheduling, e.g., via slower rate of administration and/or less frequent administration. In other embodiments, the adjusted regimen comprises administration at a dose that is moderated compared to standard scheduling, e.g., reduced dose. In other embodiments, the second anti-C5 antibody is administered at a reduced dose and/or a reduced frequency until the threshold is no longer exceeded.

In one embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered at a dose of: 600 mg weekly for four weeks, followed by 900 mg for the fifth dose one week later, then 900 mg every two weeks thereafter.

In another embodiment, the patient (e.g., adult patient) has PNH and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered at a dose of: 600 mg weekly for four weeks, followed by 900 mg for the fifth dose one week later, then 900 mg every two weeks thereafter.

In another embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the patient has aHUS and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the patient has myasthenia gravis (MG) and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., adult patient) at a dose of 900 mg weekly for four weeks, followed by 1200 mg for the fifth dose one week later, then 1200 mg every two weeks thereafter.

In another embodiment, the second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS® (eculizumab) and the adjusted regimen comprises an adjustment in the therapeutic regimen for therapy of the complement mediated disorder in the patient (e.g., an adult patient). In another embodiment, the therapeutic regimen comprises a dose of 600 mg weekly for four weeks, followed by 900 mg for the fifth dose one week later, then 900 mg every two weeks thereafter for the treatment of PNH. In another embodiment, the therapeutic regimen comprises a dose of 900 mg weekly for four weeks, followed by 1200 mg for the fifth dose one week later, then 1200 mg every two weeks thereafter for the treatment of aHUS. In another embodiment, the therapeutic regimen comprises a dose of 900 mg weekly for four weeks, followed by 1200 mg for the fifth dose one week later, then 1200 mg every two weeks thereafter for the treatment of MG.

In another embodiment, the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient (e.g., a pediatric patient) at a dose of:
(f) 900 mg weekly for four weeks, followed by 1200 mg for the fifth dose one week later, then 1200 mg every two weeks thereafter, to a patient weighing 40 kg≥;
(g) 600 mg weekly for two weeks, followed by 900 mg for the third dose one week later, then 900 mg every two weeks thereafter to a patient weighing 30 kg to <40 kg;
(h) 600 mg weekly for two weeks, followed by 600 mg for the third dose one week later, then 600 mg every two weeks thereafter, to a patient weighing 20 kg to <30;
(i) 600 mg weekly for one week, followed by 300 mg for the second dose one week later, then 300 mg every two weeks thereafter, to a patient weighing 10 kg to <20 kg; or
(j) 300 mg weekly for one week, followed by 300 mg for the second dose one week later, then 300 mg every three weeks thereafter, to a patient weighing 5 kg to <10 kg.

In another embodiment, the patient (e.g., a pediatric patient) has aHUS and the first or second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS®, administered to the patient at a dose of:
(f) 900 mg weekly for four weeks, followed by 1200 mg for the fifth dose one week later, then 1200 mg every two weeks thereafter, to a patient weighing 40 kg≥,
(g) 600 mg weekly for two weeks, followed by 900 mg for the third dose one week later, then 900 mg every two weeks thereafter to a patient weighing 30 kg to <40 kg;
(h) 600 mg weekly for two weeks, followed by 600 mg for the third dose one week later, then 600 mg every two weeks thereafter, to a patient weighing 20 kg to <30;
(i) 600 mg weekly for one week, followed by 300 mg for the second dose one week later, then 300 mg every two weeks thereafter, to a patient weighing 10 kg to <20 kg; or
(j) 300 mg weekly for one week, followed by 300 mg for the second dose one week later, then 300 mg every three weeks thereafter, to a patient weighing 5 kg to <10 kg.

In another embodiment, the second anti-C5 antibody (a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively; (b) comprises a heavy chain variable region depicted in SEQ ID NO:7 and a light chain variable region depicted in SEQ ID NO:8; (c) comprises a heavy chain polypeptide comprising the amino acid sequence depicted in SEQ ID NO:10 and a light chain polypeptide comprising the amino acid sequence depicted in SEQ ID NO:11; and/or (d) is SOLIRIS® (eculizumab) and the adjusted regimen comprises an adjustment in the therapeutic regimen for therapy of the complement mediated disorder in the patient (e.g., a pediatric patient). In another embodiment, the therapeutic regimen comprises administration of the second antibody to the patient at a dose of:
(f) 900 mg weekly for four weeks, followed by 1200 mg for the fifth dose one week later, then 1200 mg every two weeks thereafter, to a patient weighing 40 kg≥;
(g) 600 mg weekly for two weeks, followed by 900 mg for the third dose one week later, then 900 mg every two weeks thereafter to a patient weighing 30 kg to <40 kg;
(h) 600 mg weekly for two weeks, followed by 600 mg for the third dose one week later, then 600 mg every two weeks thereafter, to a patient weighing 20 kg to <30;
(i) 600 mg weekly for one week, followed by 300 mg for the second dose one week later, then 300 mg every two weeks thereafter, to a patient weighing 10 kg to <20 kg; or
(j) 300 mg weekly for one week, followed by 300 mg for the second dose one week later, then 300 mg every three weeks thereafter, to a patient weighing 5 kg to <10 kg.

In another embodiment, the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO: 8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient (e.g., an adult patient) (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the patient (e.g., adult patient) has PNH of aHUS and the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), and the adjusted regimen comprises an adjustment in the therapeutic regimen for therapy of the complement mediated disorder in the patient (e.g., an adult patient). In another embodiment, the therapeutic regimen comprises administration of the second antibody to the patient at a dose of: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

In another embodiment, the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO: 8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient (e.g., a pediatric patient): (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the patient (e.g., pediatric patient) has PNH of aHUS and the first or second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), administered to the patient: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the second anti-C5 antibody (a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; (b) comprises a heavy chain variable region depicted in SEQ ID NO:12 and a light chain variable region depicted in SEQ ID NO:8; (c) comprises a heavy chain polypeptide comprising the amino acid sequence depicted in SEQ ID NO: 14 and a light chain polypeptide comprising the amino acid sequence depicted in SEQ ID NO: 11; (d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or (e) is ULTOMIRIS® (ravulizumab), and the adjusted regimen comprises an adjustment in the therapeutic regimen for therapy of the complement mediated disorder in the patient (e.g., pediatric patient). In another embodiment, the therapeutic regimen comprises administration of the second antibody to the patient (e.g., pediatric patient) at a dose of: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and (b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

### VI. Methods of Treatment

In one embodiment, a method for treating a patient suffering from a complement mediated disorder is provided, wherein the patient has been or is being treated with a first anti-C5 antibody, and wherein the method comprises:
(a) contacting a biological sample from the patient with a therapeutic dose of a second anti-C5 antibody under conditions sufficient for formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering to the patient whose measured level exceeds the threshold level, an adjusted regimen of the second anti-C5 antibody, wherein the adjusted regimen comprises adjustment of the dose and/or timing of administration of the second anti-C5 antibody, such that C5 inhibition is maintained, but the threshold level is not exceeded.

In some embodiments, the methods further comprise weaning (e.g., withdrawing) the patient from treatment with the first anti-C5 antibody therapy.

### VII. Methods of Determining Adjusted Regimens

Also provided herein are methods for determining an adjusted regimen antibody (e.g., a regimen to prevent or minimize formation of multivalent immune complexes) comprising adjusted therapeutic dose and/or timing of administration of a second anti-C5 to treat a patient suffering from a complement mediated disorder, wherein the patient has been or is being treated with a first anti-C5 antibody, the method comprising:
(a) contacting a biological sample from the patient with a therapeutic dose of the second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) adjusting the regimen of the second anti-C5 antibody therapy to the patient whose measured level exceeds the threshold level, such that C5 inhibition is maintained, but the threshold level is not exceeded.

In some embodiments, the methods further comprise weaning (e.g., withdrawing) the patient from treatment with the first anti-C5 antibody therapy.

### VIII. Methods for Switching a Patient from a First C5-Antibody to a Second C5-Antibody

Further provided herein are methods for switching a patient having a complement mediated disorder who has been or is being treated with a first anti-C5 antibody to treatment with a second anti-C5 antibody. In one embodiment, a method for switching a patient having a complement mediated disorder who has been or is being treated with a first anti-C5 antibody to a second anti-C5 antibody is provided, the method comprising:
(a) contacting a biological sample from the patient with a therapeutic dose of a second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering, to the patient, an adjusted regimen of the second anti-C5 antibody, wherein the adjusted regimen comprises an adjustment in the dose and/or timing of administration of the second anti-C5 antibody, such that C5 inhibition is maintained, but the threshold level is not exceeded; and
(e) weaning the patient whose measured level exceeds the threshold level, from treatment with the first anti-C5 antibody therapy, thereby switching the patient from the first anti-C5 antibody to the second anti-C5 antibody.

In some embodiments of the methods described herein, the second anti-C5 antibody is administered at a reduced dose and/or a reduced frequency until the threshold is no longer exceeded.

In some embodiments of the methods described herein, the adjusted regimen comprises a modification of a clinically effective dosing or scheduling regimen. For example, in one embodiment, the adjusted regimen is a dose which is lower than a standard therapeutic dose, e.g., a sub-therapeutic dose. In other embodiments, the adjusted regimen comprises administration at a rate or interval that is moderated compared to standard scheduling, e.g., via slower rate of administration and/or less frequent administration.

### IX. Additional Treatments

Additional techniques can be used in combination with the methods described herein to clear or enhance clearance of the first anti-C5 antibody before switching to treatment with a second anti-C5 antibody. Exemplary techniques include, but are not limited to, plasmapheresis or blood transfusions.

### X. Outcomes

The efficacy of the treatment methods provided herein can be assessed using any suitable means. In one embodiment, the treatment resolves at least one sign or symptom of the complement-mediated disorder without toxicity associated with multivalent immune complexes formed as a result of administration of the second anti-C5 antibody.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLE

### Example 1

The properties of circulating immune complexes formed with target antigens should be considered when developing a monoclonal antibody (mAb). Valency of such complexes can affect clearance, effector function and uptake by phagocytic cells. Therefore, consideration must be given when a recipient is exposed to more than one immunoglobulin G antibody that bind to a monovalent target at different sites, potentially leading to the formation of multivalent immune complexes. Accordingly, the objective of the present study is to better understand the potential for formation of multivalent complexes in recipients of other non-competitive anti-C5 antibodies, while maintaining detectable levels of eculizumab in circulation.

1. Methods

Size exclusion chromatography (SEC), in combination with multi-angle light scattering (MALS), was used to assess the size distribution of ternary complexes formed in vitro between eculizumab, C5 and two other anti-C5 monoclonal antibodies that have the same sequences as mAbs currently undergoing clinical trials, but which bind to different C5 epitopes. mAb crovalimab mimetic was generated using the sequences of a monoclonal antibody now known as crovalimab (Chugai Pharmaceutical, Tokyo, Japan). pozelimab mimetic was based on the sequences of the monoclonal antibody now known as pozelimab (Regeneron Pharmaceuticals, New York, NY, USA).

Eculizumab was labeled with Alexa Fluor 488 Dye (Thermo Fisher Scientific, Waltham, MA, USA). The competition between anti-C5 mAbs was evaluated using bio-layer interferometry.

Hemolysis was assessed by the release of soluble hemoglobin determined at 415 nm, following 30-minute incubations at 37° C. of varying concentrations of anti-C5 monoclonal antibodies with 2.5×106 antibody sensitized chicken red blood cells in gelatin veronal buffered saline containing 0.15 mM CaCl2 and 0.5 mM MgCl2 (GVB++) and 15% (v/v) normal human serum. All C5 monoclonal antibodies were functional and had comparable half maximal inhibitory concentration (IC50) values (data not shown).

To identify, characterize, and determine the masses of immune complexes, purified eculizumab, human C5 and either crovalimab mimetic or pozelimab mimetic were incubated together in varying molar ratios in phosphate buffered saline (PBS) for ˜30 minutes at room temperature. Samples were placed at 2-8° C. in a refrigerated autoinjector for high performance liquid chromatography (1260 LC System, Agilent, Santa Clara, CA, USA) and applied (45 μL/injection) in sequence to a 4.6×300 mm Bio SEC-5 Column (Agilent) complete with upstream in-line 0.2 μm filter, and previously equilibrated and run using PBS as mobile phase at 0.3 mL/minute. Eluent was monitored at absorbance 280 nm and an in-line MALS detector (Treos-II, Wyatt Technology, Santa Barbara, CA, USA) with a differential refractive index detector (Optilab T-rEX, Wyatt Technology) was used to quantify molecular masses. To evaluate the potential for such complexes to form in human plasma, eculizumab was replaced with labeled eculizumab and the incubations were performed in at least 80% (v/v) final human plasma with 35 μL injections onto the SEC column. The same chromatography method as described above for samples diluted in PBS was used, except that chromatography was monitored with in-line fluorescence detection using a fluorescence detector (Agilent), in which elution was monitored with excitation at 490 nm and emission at 525 nm.

2. Results

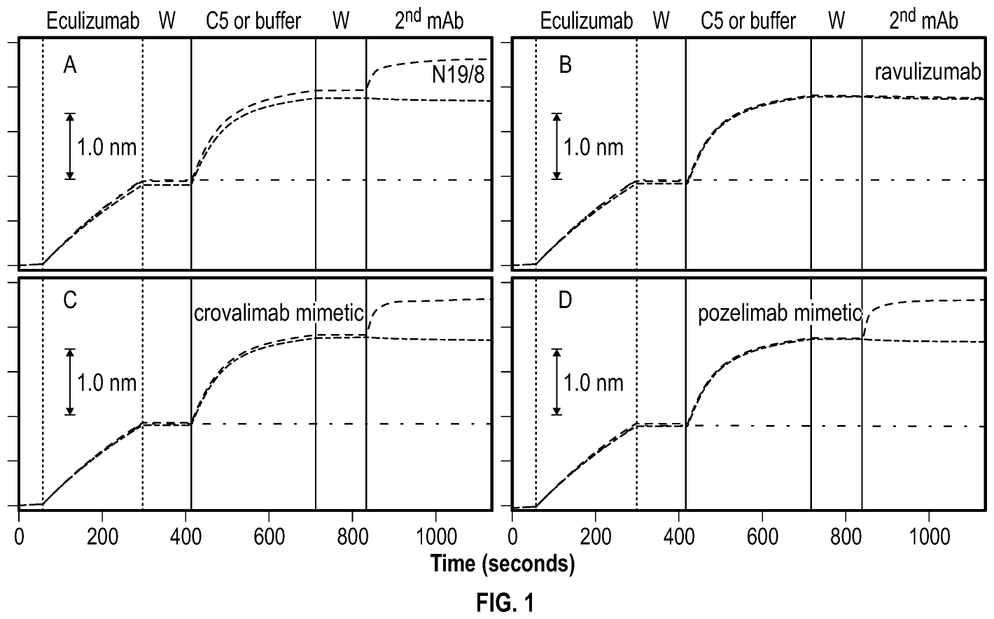

Biotinylated eculizumab was captured onto a streptavidin biosensor probe, washed (W) and treated with either purified human C5 (red and black traces) or buffer (blue traces) (See FIGS. 1A-1D). Following an additional wash, probes, were treated with either eculizumab (black traces), or a different second antibody (red traces) or buffer (blue traces) (see FIGS. 1A-1D). FIGS. 1A-D set forth the data for the following different second antibodies: FIG. 1A: mAb N19/8 which is known to bind to a different site on C5 to eculizumab; FIG. 1B: ravulizumab; FIG. 1C: mAb crovalimab mimetic; and FIG. 1D: mAb pozelimab mimetic. The scale bar shows the deflection corresponding to a biosensor signal of 1.0 nm. mAb, monoclonal antibody. In sum, recombinant monoclonal antibodies crovalimab mimetic and pozelimab mimetic were each functional and potent inhibitors of in vitro classical pathway hemolysis (data not shown), and both monoclonal antibodies could associate non-competitively with eculizumab-bound C5.

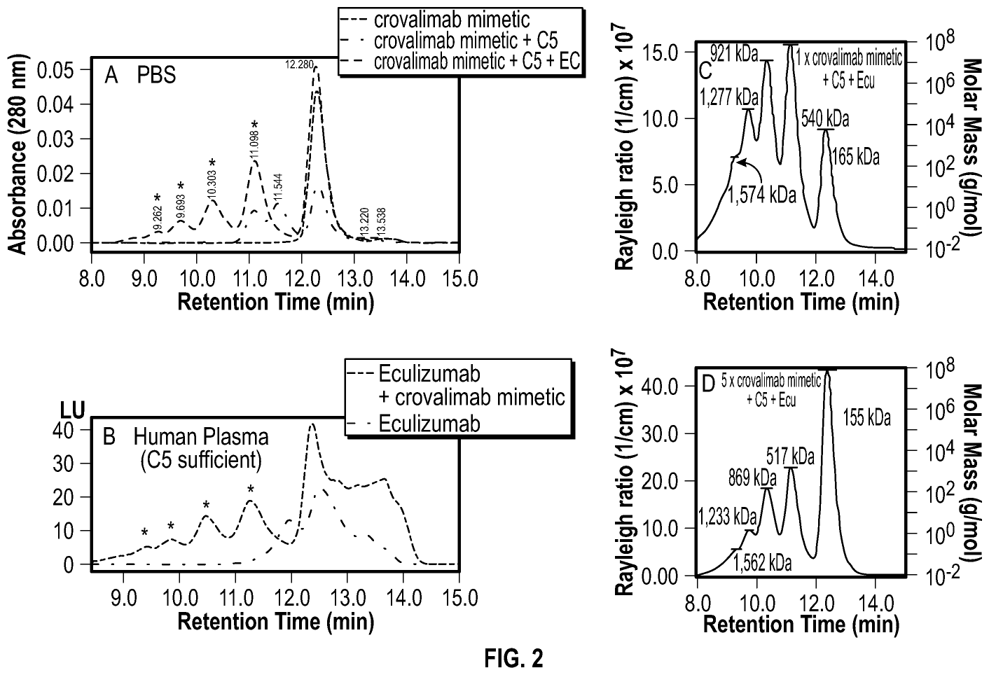

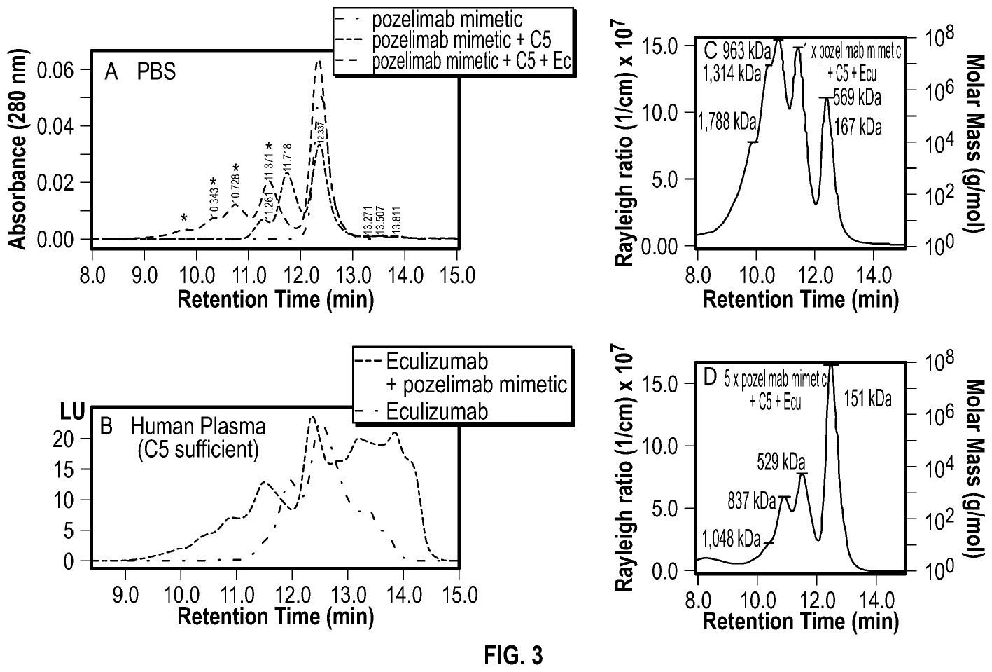

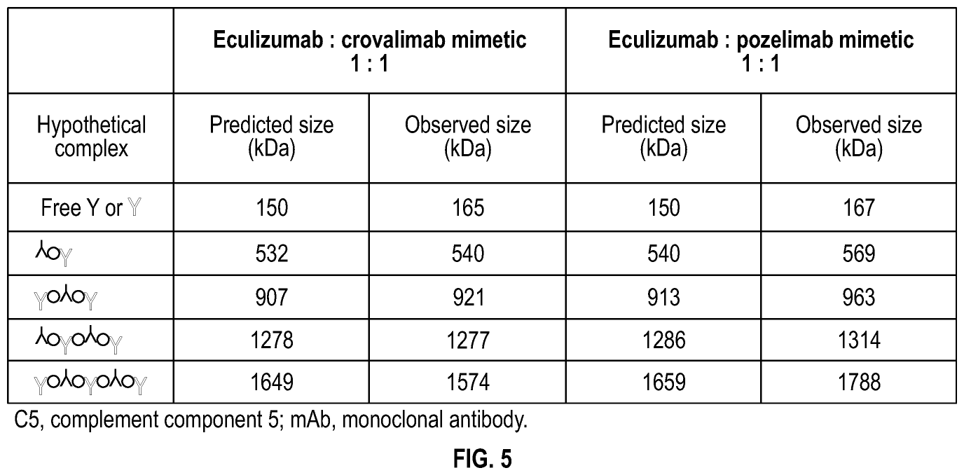

Compositions in PBS comprising either crovalimab mimetic (FIGS. 2A and C) or pozelimab mimetic (FIGS. 3A and C) together with C5 plus eculizumab at levels expected in vivo as eculizumab approaches the minimum plasma concentration (Cₘᵢₙ), contained a series of high molecular weight complexes with some larger than 1500 kDa, and were consistent with incorporation of up to four molecules of C5 and five molecules of mAb (FIG. 5).

Large complexes with similar migration in SEC were observed with crovalimab mimetic (FIG. 2B) or pozelimab mimetic (FIG. 3B), when these compositions were more than 80% plasma. Large complexes were observed both when crovalimab mimetic (FIGS. 2C and D) or pozelimab mimetic (FIGS. 3C and D) were equimolar or in fivefold excess of eculizumab.

More specifically, as shown in FIG. 2A, crovalimab mimetic (700 nM) alone migrated as a single peak and formed two additional species in the presence of C5 (400 nM) corresponding to 1:1 and 1:2 crovalimab mimetic:C5 complexes (MALS data not shown). Reconstitution of crovalimab mimetic (700 nM) with eculizumab (700 nM) plus C5 (400 nM) resulted in the formation of a series of larger, earlier eluting peaks denoted with asterisks. As shown in FIG. 2B, earlier eluting complexes with highly similar retention times (*) were observed when crovalimab mimetic (700 nM) was reconstituted with labeled eculizumab (700 nM) in 80% human plasma, but not when crovalimab mimetic was absent. Complexes formed between crovalimab mimetic, C5 and eculizumab in PBS were in some cases greater than 1.5 million Da by MALS irrespective of whether reconstitution was with a concentration of crovalimab mimetic equimolar (700 nM) to eculizumab (FIG. 2C) or fivefold molar excess (3500 nM) of eculizumab (FIG. 2D).

In sum, crovalimab mimetic induces formation of very large complexes in the presence of eculizumab and C5 at eculizumab and C5 concentrations mimicking those in human blood as eculizumab approaches Cₘᵢₙ.

In addition, as shown in FIGS. 3A-3D, pozelimab mimetic induces formation of very large complexes in the presence of eculizumab plus C5 at concentrations of eculizumab and C5 mimicking those in human blood as eculizumab approaches Cₘᵢₙ. Specifically, as shown in FIG. 3A, pozelimab mimetic (700 nM) alone migrated as a single peak and formed two additional species in the presence of C5 (400 nM) corresponding to 1:1 and 1:2 pozelimab mimetic:C5 complexes (MALS data not shown). Reconstitution of pozelimab mimetic (700 nM) with eculizumab (700 nM) plus C5 (400 nM) resulted in the formation of a series of larger, earlier eluting peaks denoted with asterisks. As shown in FIG. 3B, earlier eluting complexes with highly similar retention times (*) were also observed when pozelimab mimetic (700 nM) was reconstituted with labeled eculizumab (700 nM) in 80% human plasma, but not when pozelimab mimetic was absent. Complexes formed between pozelimab mimetic, C5 and eculizumab in PBS were in some cases greater than 1.5 million Da by MALS (FIG. 3C) and remained very large even when pozelimab mimetic was at 3500 nM, a fivefold excess to eculizumab (FIG. 3D).

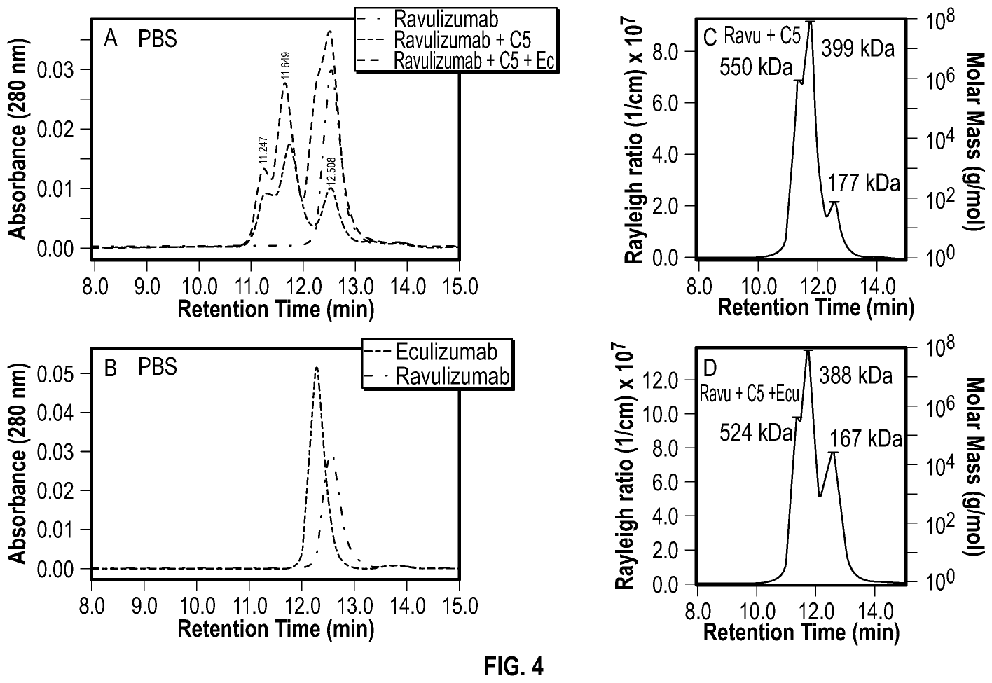

However, ravulizumab did not associate with eculizumab-bound C5 (FIG. 1B) and did not form complexes beyond the sizes expected for 1:1 and 1:2 mAb:C5 complexes (FIGS. 4A-4D). Ravulizumab forms only 1:1 and 1:2 complexes with C5 both in the absence or presence of eculizumab. Specifically, ravulizumab alone migrated as a single peak, but in the presence of C5 or C5 plus eculizumab three peaks were observed. The last peak to elute in the composition of C5 plus eculizumab and ravulizumab corresponds to unbound antibody and had a shoulder because free eculizumab and ravulizumab had slightly different retention times (FIG. 4B). The compositions comprising ravulizumab and C5 (FIG. 4C), and ravulizumab and C5 plus eculizumab (D) exhibited masses by multi-angle light scattering (MALS) consistent with the formation of only 1:1 and 1:2 mAb:C5 complexes. No complexes larger than these were observed.

3. Conclusions

These experiments demonstrate that multivalent complexes can be formed from the combination of C5 with two monoclonal antibodies binding different C5 epitopes. These multivalent complexes have unknown pharmacologic properties, and the safety of such complexes if formed in vivo is presently unknown. Accordingly, these findings have potential implications for when a recipient may be simultaneously exposed to more than one, non-competing, anti-C5 monoclonal antibody, such as when a patient is converted from one such antibody to another, and where continued maintenance of PD suppression is essential.

This study highlights the importance of understanding the nature and properties of such complexes, and the incorporation of monitoring and mitigation processes to assess and prevent their potential formation in patients. If formed in vivo, such complexes could potentially be proinflammatory via one or more of the mechanisms associated with polyvalent immune complexes, such as: avidity-driven reconstitution of Fcγ or C1q dependent effector functions, deposition of complexes in tissues, altered antigen processing and immunogenicity directed against the constituents associated with polyvalent interaction with the neonatal Fc receptor (FcRn), neutrophil activation, cytokine release, degranulation and NETosis.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SS
SEQ ID NO: 8
DIQMTQSPSS LSASVGDRVT ITCGASENIY GALNWYQQKP GKAPKLLIYG
ATNLADGVPS RESGSGSGTD FTLTISSLQP EDFATYYCQN VLNTPLTFGQ
GTKVEIK
SEQ ID NO: 9
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVMHEA LHNHYTQKSL SLSLGK
SEQ ID NO: 10
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYE
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS
DGS FFLYSRL TVDKSRWQEG NVESCSVMHE ALHNHYTQKS LSLSLGK
SEQ ID NO: 11
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTFGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSEN RGEC
SEQ ID NO: 12
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SS
SEQ ID NO: 13
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVLHEA LHSHYTQKSL SLSLGK
SEQ ID NO: 14
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVLH EALHSHYTQK SLSLSLGK
SEQ ID NO: 15
ASTKGPSVFP LAPCSRSTSE STAALGCLVK DYFPEPVTVS WNSGALTSGV
HTFPAVLQSS GLYSLSSVVT VTSSNFGTQT YTCNVDHKPS NTKVDKTVER
KCCVECPPCP APPVAGPSVF LFPPKPKDTL YITREPEVTC VVVDVSHEDP
EVQFNWYVDG MEVHNAKTKP REEQFNSTFR VVSVLTVVHQ DWLNGKEYKC
KVSNKGLPAP IEKTISKTKG QPREPQVYTL PPSREEMTKN QVSLTCLVKG
FYPSDIAVEW ESNGQPENNY KTTPPMLDSD GSFFLYSKLT VDKSRWQQGN
VFSCSVMHEA LHNHYTQKSL SLSPGK
SEQ ID NO: 16
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVTSSNFGT
QTYTCNVDHK PSNTKVDKTV ERKCCVECPP CPAPPVAGPS VFLFPPKPKD
TLYITREPEV TCVVVDVSHE DPEVQFNWYV DGMEVHNAKT KPREEQENST
FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD
SDGSFFLYSK LTVDKSRWQQ GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWINGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTFS SYAISVWRQA PGQGLEWMGG
IGPFFGTANY AQKFQGRVTI TADESTSTAY MELSSLRSED TAVYYCARDT
PYFDYWGQGT LVTVSS
SEQ ID NO: 28
DIELTQPPSV SVAPGQTARI SCSGDSIPNY YVYWYQQKPG QAPVLVIYDD
SNRPSGIPER FSGSNSGNTA TLTISGTQAE DEADYYCQSF DSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAE VKKPGESLKI SCKGSGYSFT NYISWVRQMP GKGLEWMGII
DPDDSYTEYS PSFQGQVTIS ADKSISTAYL QWSSLKASDT AMYYCARYEY
GGFDIWGQGT LVTVSS
SEQ ID NO: 36
SYELTQPPSV SVAPGQTARI SCSGDNIGNS YVHWYQQKPG QAPVLVIYKD
NDRPSGIPER FSGSNSGNTA TLTISGTQAE DEADYYCGTY DIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGG LVQPGGSLRL SCAASGFTSH SSYYVAWVRQ APGKGLEWVG
AIYTGSGATY KASWAKGRFT ISKDTSKNQV VLTMTNMDPV DTATYYCASD
GGYDYPTHAM HYWGQGTLVT VSS
SEQ ID NO: 44
DVVMTQSPSS LSASVGDRVT ITCQASQNIG SSLAWYQQKP GQAPRLLIYG
ASKTHSGVPS RFSGSGSGTD FTLTISSLQP EDVATYYCQS TKVGSSYGNH
FGGGTKVEIK
SEQ ID NO: 45
QVQLVESGGG LVQPGRSLRL SCAASGFTVH SSYYMAWVRQ APGKGLEWVG
AIFTGSGAEY KAEWAKGRVT ISKDTSKNQV VLTMTNMDPV DTATYYCASD
AGYDYPTHAM HYWGQGTLVT VSSASTKGPS VFPLAPSSKS TSGGTAALGC
LVKDYFPEPV TVSWNSGALT SGVHTFPAVL QSSGLYSLSS VVTVPSSSLG
TQTYICNVNH KPSNTKVDKK VEPKSCDKTH TCPPCPAPEL RRGPKVFLFP
PKPKDTLMIS RTPEVTCVVV DVSHEDPEVK FNWYVDGVEV HNAKTKPREE
QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT
PPVLDSDGSF FLYSKLTVDK SRWQQGNVES CSVLHEALHA HYTRKELSLS
P
SEQ ID NO: 46
DIQMTQSPSS LSASVGDRVT ITCRASQGIS SSLAWYQQKP GKAPKLLIYG
ASETESGVPS RFSGSGSGTD FTLTISSLQP EDFATYYCQN TKVGSSYGNT
FGGGTKVEIK RTVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SENRGEC
SEQ ID NO: 47
QVQLQESGPGLVKPSETLSLTCTVSGDSVSSSYWTWIRQPPGKGLEWIGYIYYSGSSN
YNPSLKSRATISVDTSKNQFSLKLSSVTAADTAVYYCAREGNVDTTMIFDYWGQGTLV
TVSS
SEQ ID NO: 48
AIQMTQSPSSLSASVGDRVTITCRASQGIRNDLGWYQQKPGKAPKLLIYAASSLQSGVP
SRFAGRGSGTDFTLTISSLQPEDFATYYCLQDENYPWTFGQGTKVEIK
SEQ ID NO: 49
QVQLQESGPGLVKPSETLSLTCTVSGDSVSSSYWTWIRQPPGKGLEWIGYIYYSGSSNY
NPSLKSRATISVDTSKNQFSLKLSSVTAADTAVYYCAREGNVDTTMIFDYWGQGTLVTV
SSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSENRGEC

What is claimed is:

1. A method for treating a patient suffering from a complement mediated disorder, wherein the patient has been or is being treated with a first anti-C5 antibody, the method comprising:
(a) contacting a biological sample from the patient with a therapeutic dose of a second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering to the patient whose measured level exceeds the threshold level, an adjusted regimen of the second anti-C5 antibody, wherein the adjusted regimen comprises adjustment of the dose and/or timing of administration of the second anti-C5 antibody, such that C5 inhibition is maintained, but the threshold level is not exceeded.

2. A method for determining an adjusted regimen comprising adjusted therapeutic dose and/or timing of administration of a second anti-C5 antibody to treat a patient suffering from a complement mediated disorder, wherein the patient has been or is being treated with a first anti-C5 antibody, the method comprising:
(a) contacting a biological sample from the patient with a therapeutic dose of the second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) adjusting the regimen of the second anti-C5 antibody therapy to the patient whose measured level exceeds the threshold level, such that C5 inhibition is maintained, but the threshold level is not exceeded.

3. The method of claim 1 or 2, further comprising weaning the patient from treatment with the first anti-C5 antibody therapy.

4. A method for switching a patient having a complement mediated disorder who has been or is being treated with a first anti-C5 antibody to a second anti-C5 antibody, the method comprising:
(a) contacting a biological sample from the patient with a therapeutic dose of a second anti-C5 antibody under conditions sufficient for the formation of multivalent immune complexes comprising complement C5, the first anti-C5 antibody, and second anti-C5 antibody;
(b) measuring a level of multivalent immune complexes formed under (a);
(c) determining if the measured level of multivalent immune complexes exceeds a threshold level; and
(d) administering, to the patient, an adjusted regimen of the second anti-C5 antibody, wherein the adjusted regimen comprises an adjustment in the dose and/or timing of administration of the second anti-C5 antibody, such that C5 inhibition is maintained, but the threshold level is not exceeded; and
(d) weaning the patient whose measured level exceeds the threshold level, from treatment with the first anti-C5 antibody therapy, thereby switching the patient from the first anti-C5 antibody to the second anti-C5 antibody.

5. The method of any of the preceding claims, wherein the second anti-C5 antibody is administered at a reduced dose and/or a reduced frequency until the threshold is no longer exceeded.

6. The method of any of the preceding claims, wherein the adjusted regimen comprises:
a) a modification of a clinically effective dosing or scheduling regimen; and/or
b) administration at a rate and/or interval that is moderated compared to standard scheduling.

7. The method of claim 6, wherein the modification is administration of a dose which is lower than a standard therapeutic dose.

8. The method of claim 6, wherein the administration comprises a slower rate of administration and/or less frequent administration.

9. The method of any of the preceding claims, wherein the threshold value consists of a minimum mass of the multivalent immune complexes.

10. The method of any of the preceding claims, wherein the threshold value is a mass of more than 500 kDa, 600 kDa, 700 kDa, 800 kDa, 900 kDa, 1000 kDa, 1100 kDa, 1200 kDa, 1300 kDa, 1400 kDa, 1500 kDa, 1600 kDa, 1700 kDa, or 1800 kDa.

11. The method of any of claims 1-8, wherein the threshold value is formation of multivalent immune complexes comprising more than two anti-C5 antibody molecules bound specifically to a molecule of complement C5, e.g., immune complexes comprising antibody:antigen stoichiometric ratios exceeding:
a) 2 anti-C5 antibodies and 1 C5 molecule;
b) 3 anti-C5 antibodies and 2 C5 molecules;
c) 4 anti-C5 antibodies and 3 C5 molecules; or
d) 5 anti-C5 antibodies and 4 C5 molecules.

12. The method of any of the preceding claims, wherein the threshold level is determined by size exclusion chromatography (SEC) and/or multi-angle light scattering (MALS).

13. The method of any of the preceding claims, wherein in step (a), the biological sample from the patient is contacted with a therapeutic dose of a second anti-C5 antibody in vivo, ex vivo, or in vitro.

14. The method of any of the preceding claims, wherein if the threshold is exceeded, administration of the second anti-C5 antibody to the patient is deferred by 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days to allow for clearance of the first anti-C5 antibody from the patient.

15. The method of any of the preceding claims, wherein the first and/or second anti-C5 antibodies are selected from the group consisting of full-length antibody or an antigen-binding fragment thereof, a humanized antibody, bispecific antibody, an immunoconjugate, a chimeric antibody, a protein scaffold with antibody-like properties, such as fibronectin or ankyrin repeats, a Fab, Fab′2, scFv, affibody, avimer, nanobody, and a domain antibody.

16. The method of any of the preceding claims, wherein the first and/or second anti-C5 antibody is a monoclonal antibody.

17. The method of any of the preceding claims, wherein the first and second anti-C5 antibodies are antibodies that bind different epitopes on C5 or antibodies that do not compete for binding to C5, preferably wherein the first and/or the second anti-C5 antibodies are monoclonal antibodies; more preferably wherein the first and/or the second anti-C5 antibodies are monoclonal antibodies that bind to different domains in C5.

18. The method of any of the preceding claims, wherein the first or second anti-C5 antibody:
(a) comprises CDR1, CDR2 and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively;
(b) comprises a heavy chain variable region depicted in SEQ ID NO:7 and a light chain variable region depicted in SEQ ID NO:8;
(c) comprises a heavy chain polypeptide comprising the amino acid sequence depicted in SEQ ID NO:10 and a light chain polypeptide comprising the amino acid sequence depicted in SEQ ID NO:11; and/or
(d) is SOLIRIS® (eculizumab).

19. The method of claim 18, wherein the first or second anti-C5 antibody comprises the antibody of (a)-(d) and the adjusted regimen comprises an adjustment in the therapeutic regimen for the therapy of the complement mediated disorder in an adult patient.

20. The method of claim 18 or 19, wherein the therapeutic regimen comprises a dose of:
(a) 600 mg weekly for four weeks, followed by 900 mg for the fifth dose one week later, then 900 mg every two weeks thereafter for the treatment of PNH; or
(b) 900 mg weekly for four weeks, followed by 1200 mg for the fifth dose one week later, then 1200 mg every two weeks thereafter for the treatment of aHUS or MG.

21. The method of claim 18, wherein the first or second anti-C5 antibody comprises the antibody of (a)-(d) and the adjusted regimen comprises an adjustment in the therapeutic regimen of the second antibody for the therapy of the complement mediated disorder in a pediatric patient.

22. The method of claim 18 or 21, wherein the therapeutic regimen comprises administration of the following doses of the first or second anti-C5 antibody:
(a) to an aHUS patient weighing 40 kg≥, a dose of 900 mg weekly for four weeks, followed by 1200 mg for the fifth dose one week later, then 1200 mg every two weeks thereafter;
(b) to an aHUS patient weighing 30 kg to <40 kg, a dose of 600 mg weekly for two weeks, followed by 900 mg for the third dose one week later, then 900 mg every two weeks thereafter;
(c) to an aHUS patient weighing 20 kg to <30 kg a dose of 600 mg weekly for two weeks, followed by 600 mg for the third dose one week later, then 600 mg every two weeks thereafter;
(d) to an aHUS patient weighing 10 kg to <20 kg, a dose of 600 mg weekly for one week, followed by 300 mg for the second dose one week later, then 300 mg every two weeks thereafter; or
(e) to an aHUS patient weighing 5 kg to <10 kg, a dose of 300 mg weekly for one week, followed by 300 mg for the second dose one week later, then 300 mg every three weeks thereafter.

23. The method of any of claims 1-17, wherein the first or second anti-C5 monoclonal antibody:
(a) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively;
(b) comprises a heavy chain variable region depicted in SEQ ID NO:12 and a light chain variable region depicted in SEQ ID NO:8;
(c) comprises a heavy chain polypeptide comprising the amino acid sequence depicted in SEQ ID NO: 14 and a light chain polypeptide comprising the amino acid sequence depicted in SEQ ID NO: 11;
(d) comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a heavy chain constant region as set forth in SEQ ID NO:13 and/or
(e) is ULTOMIRIS® (ravulizumab).

24. The method of claim 23, wherein the first or second anti-C5 antibody comprises the antibody of (a)-(e) and the adjusted regimen comprises an adjustment in the therapeutic regimen of the second antibody for the therapy of the complement mediated disorder in an adult patient.

25. The method of claim 24, wherein the therapeutic regimen of the second anti-C5 antibody comprises:
(a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and
(b) on Day 15 of the administration cycle and every eight weeks thereafter at a dose of 3000 mg to a patient weighing ≥40 to <60 kg, 3300 mg to a patient weighing ≥60 to <100 kg or 3600 mg to a patient weighing ≥100 kg.

26. The method of claim 23, wherein the first or second anti-C5 antibody comprises the antibody of (a)-(e) and the adjusted regimen comprises an adjustment in the therapeutic regimen of the second antibody for the therapy of the complement mediated disorder in a pediatric patient.

27. The method of claim 23 or 26, wherein the therapeutic regimen of the first or second anti-C5 antibody for treating PNH or aHUS in pediatric patients comprises:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; and
(b) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

28. The method of any of the preceding claims, wherein the first anti-C5 antibody is selected from the group consisting of eculizumab and ravulizumab.

29. The method of any of the preceding claims, wherein the first or the second anti-C5 antibody is selected from the group consisting of 7086 antibody, 8110 antibody, 305LO5 antibody, SKY59 antibody, and pozelimab (REGN3918 antibody), Tesidolumab (LFG316), Crovalimab (RG6107), ABP 959 antibody, ELIZARIA®, BCD-148 (JSC BIOCAD) and/or SB12 or antigen-binding fragments thereof comprising.

30. The method of any of the preceding claims, wherein the biological sample is selected from the group consisting of blood, serum, plasma, urine, saliva, lymph, spinal fluid, intercellular fluid, vitreous humor, and sweat.

31. The method of claim 30, wherein the biological sample is blood.

32. The method of any of the preceding claims, wherein the first and/or second anti-C5 antibody is administered intravenously or subcutaneously.

33. The method of any of the preceding claims, wherein the complement-associated condition is selected from the group consisting of rheumatoid arthritis, antiphospholipid antibody syndrome, lupus nephritis, ischemia-reperfusion injury, atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome.

34. The method of claim 33, wherein the complement-associated condition is paroxysmal nocturnal hemoglobinuria (PNH).

35. The method of claim 33, wherein the complement-associated condition is atypical hemolytic uremic syndrome (aHUS).

36. The method of any of the preceding claims, wherein the treatment resolves at least one sign or symptom of the complement-mediated disorder without toxicity associated with multivalent immune complexes formed as a result of administration of the second anti-C5 antibody.

37. Use of a therapeutically effective amount of a second anti-C5 antibody that is non-competitive with a first anti-C5 antibody, for the treatment of a complement-mediated disease (e.g., aHUS, PNH, HSCT-TMA, CM-TMA, NMOSD, gMG or ALS) in a patient who is being treated or has been treated with the first anti-C5 antibody, wherein the second anti-C5 antibody is administered after a washout sufficient to reduce levels of the first anti-C5 antibody in the patient's system (e.g., blood) such that formation of multivalent immune complexes comprising the first and the second anti-C5 antibodies and complement C5 is prevented or reduced.

38. Use according claim 37, wherein the multivalent immune complex comprises at least one first antibody and at least one second antibody that are specifically bound to a complement C5 antigen, and optionally, additional C5 antigens.

39. Use according claim 37, wherein the second antibody is non-competitive with the first antibody selected from eculizumab and ravulizumab or a biosimilar thereof.

40. Use according claim 37, wherein the first antibody is selected from eculizumab and ravulizumab or a biosimilar thereof and wherein the second antibody is selected from 305LO5, SKY59, pozelimab, tesidolumab, crovalimab, or ABP 959 or a biosimilar thereof; preferably, wherein the second antibody is pozelimab, tesidolumab, or crovalimab.

41. Use according claim 37, wherein the complement-mediated disorder is aHUS or PNH.


SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC